(19) United States
(12) Patent Application Publication
(10) Pub. No.: US 2016/0024581 A1
(43) Pub. Date: Jan. 28, 2016

(54) METHODS AND COMPOSITIONS FOR ASSESSING RENAL STATUS USING URINE CELL FREE DNA

(71) Applicant: IMMUCOR GTI DIAGNOSTICS, INC., Norcross, GA (US)

(72) Inventors: Minnie M. Sarwal, Mountain View, CA (US); Tara Sigdel, Mountain View, CA (US)

(21) Appl. No.: 14/774,789

(22) PCT Filed: Mar. 15, 2014
(86) PCT No.: PCT/US2014/029956

§ 371 Date: Sep. 11, 2015

Related U.S. Application Data
(60) Provisional application No. 61/793,427, filed on Mar. 15, 2013.

(51) Int. Cl. C12Q 1/68

(52) U.S. Cl. CPC C12Q 1/6883; C12Q 2600/158; C12Q 2600/112

(57) ABSTRACT

The present invention relates to non-invasive tools and methods for evaluating renal status and renal health using urine cell free DNA.

### CROSS-REFERENCE TO RELATED APPLICATION

This application claims the priority benefit to U.S. Provisional Patent Application Ser. No. 61/793,427, filed Mar. 15, 2013, the entire content of which is incorporated herein by reference.

### BACKGROUND

The kidneys, collectively known as the renal system, perform the essential function of removing waste products from the blood and regulating the water fluid levels. They are essential in the urinary system, but also serve homeostatic functions such as the regulation of electrolytes, maintenance of acid-base balance, and regulation of blood pressure. They serve the body as a natural filter of the blood, and remove wastes which are diverted to the urinary bladder. In producing urine, the kidneys excrete wastes such as urea and ammonium, and they are also responsible for the reabsorption of water, glucose, and amino acids. The kidneys also produce hormones including calcitriol, erythropoietin, and the enzyme renin.

Renal status is affected by renal disease or injury (also referred to as kidney injury, nephropathy) and can result from both acute and chronic conditions. Another form of altered renal status is the rejection of a transplanted kidney.

Classically, renal status is evaluated by using a blood test for creatinine. Higher levels of creatinine indicate a lower glomerular filtration rate and as a result a decreased capability of the kidneys to excrete waste products. To fully investigate the underlying cause of renal damage, various forms of medical imaging, blood tests and renal biopsy (removing a small sample of renal tissue) are often employed to find out if there is a reversible cause for the kidney malfunction.

There is a critical unmet need for improved non-invasive diagnosis of renal injury and renal transplant injury and for assessing renal status generally. In renal transplant injury, for example, donor derived cell free DNA (dd-cfDNA) has been shown to be a surrogate of transplant injury in blood but requires the expense of donor and recipient DNA sequencing.

More recently, cell free DNA (cfDNA) from dying cells has been discovered in human urine. Recent efforts have focused on testing of urine for cfDNA as a marker for allograft rejection, however, the technique has been limited to detecting Y chromosome-specific sequences in female recipients who have received a male kidney or by pre-determining differences in the genetic makeup/molecular markers between the donor and the recipient, and probing for those differences. As such, these types of testing are limited to certain types of configurations (e.g., female transplant recipient receiving a male donor kidney) and cannot be widely applicable to all transplant configurations.

Accordingly, improved compositions and methods for non-invasive testing that are widely applicable to all renal transplant situations are needed to evaluate renal status, renal injury or renal graft rejection using urine cfDNA. An approach that is rapid for measuring urine cell free DNA in all renal injury and renal transplant patients, irrespective of gender, and without the expense of DNA sequencing is needed.

Compositions and methods for such an approach, such as the high-throughput approach are provided herein.

All publications and patent applications cited in this specification are incorporated herein by reference as if each individual publication or patent application were specifically and individually indicated to be incorporated by reference.

### SUMMARY OF THE INVENTION

Urine cell free DNA (cfDNA) can be a powerful tool for the estimation and evaluation of renal status, renal health, renal injury, renal transplant injury and high grade acute renal rejection. Serial analysis of urine cfDNA loads can be carried out by digital or standard qPCR, without the need for donor and recipient DNA sequencing.

The present invention relates to using cfDNA found in the urine to evaluate renal status and renal health of the individual. Specifically, the present invention relates to methods and compositions for monitoring, diagnosis, prognosis, and evaluation of treatment regimens in subjects suffering from or suspected of having an altered renal status.

This invention provides for, inter alia, compositions and methods for assessing renal status, renal injury or renal graft rejection, which are based on measuring cfDNA of autosomal chromosome in urine.

In one aspect, the invention provides a method for assessing renal status in an individual comprising: determining the copy number of an autosomal chromosome in a urine sample, and comparing the copy number of said chromosome to a standard copy number of said chromosome in a urine sample from a normal population, wherein a change in the copy number is indicative of an altered renal status. In one embodiment, the autosomal chromosome is chromosome 1. In one embodiment, the copy number of chromosome 1 is measured using the EIF2C1 locus. In one embodiment, the EIF2C1 locus is measured using a primer set comprising a forward primer 5′-GTTCGGCTTTCACCAGTCT and a reverse primer 5′-CTCCATAGCTCTCCCACTC. In one embodiment, the primer set is utilized for real-time PCR and further includes a probe. In one embodiment, the probe comprises the sequence and reporters as follows: 5′-HEX-CGCCCTGCCATGTGGAAGAT-BHQ1.

In any of the embodiments herein, a copy number determined to be higher than the standard copy number is indicative of compromised renal status. In any of the embodiments herein, a copy number determined to be equal or lower than the standard copy number is indicative good renal health. In any of the embodiments herein, the copy number is determined using real-time PCR, quantitative PCR, or digital PCR (dPCR). In any of the embodiments herein, the copy number is determined using the BioMark real-time PCR system. In any of the embodiments herein, a compromised renal status can comprise renal damage, renal injury, a renal disease, a renal disorder, renal graft rejection, or being non-responsive to a treatment for renal damage, renal injury, renal disease, renal disorder, or renal graft rejection. In any of the embodiments herein, the assessment of renal status can comprise measuring the progression of a renal disease, a renal injury, a renal graft injury, or a renal graft rejection. In any of the embodiments herein, the assessment of renal status can comprise measuring treatment response in and individual who is suffering from a renal disease, a renal injury, a renal graft injury, or a renal graft rejection and is currently undergoing or has undergone treatment.

In any of the embodiments herein, cfDNA can be extracted from the urine. In any of the embodiments herein, the copy number is determined in cfDNA extracted from the urine In any one of the embodiments herein, the method can further comprise measurement of other pathological and clinical data. In any one of the embodiments herein, the method can further comprise measuring the amount of creatinine. In any one of the embodiments herein, the method can further comprise measuring proteinuria or cGFR. In any one of the embodiments herein, the method can further comprise performing a renal biopsy.

In any one of the embodiments herein, the individual is an individual at risk for renal damage, renal injury, a renal disease, a renal disorder, renal graft injury, or renal graft rejection. In any one of the embodiments herein, the individual is diabetic. In any one of the embodiments herein, the individual suffers from hypertension. In any one of the embodiments herein, the individual is a recipient of an allograft renal transplant. In any one of the embodiments herein, the individual is under treatment for renal graft injury or renal graft rejection. In any one of the embodiments herein, the individual has suffered at least one acute rejection episode.

In another aspect, the invention provides for a method for assessing renal status in an individual comprising: determining the number of ALU repeats in a urine sample, and comparing the number of ALU repeats to a standard number of ALU repeats in a urine sample from a normal population, wherein a change in the number of ALU repeats is indicative of an altered renal status. In one embodiment, a number of ALU repeats determined to be higher than the standard number of repeats is indicative of compromised renal status. In one embodiment, the number of ALU repeats determined to be equal or lower than the standard number of ALU repeats is indicative good renal health. In another embodiment, the number of ALU repeats is determined in cfDNA extracted from the urine. In another embodiment, the number of ALU repeats is determined using real-time PCR, quantitative PCR, or digital PCR. In another embodiment, the number of ALU repeats is determined using the BioMark real-time PCR system. In another embodiment, the number of ALU repeats is determined using real-time PCR. In another embodiment, the number of ALU repeats is measured using a 115 base amplicon of the ALU locus. In any of the embodiments herein, the method can further comprise measuring the number of copies of an autosomal chromosome in the same sample. In another embodiment, the copy number of chromosome 1 is measured. In one embodiment, the copy number of Chromosome 1 is measured using the EIF2C1 locus. In another embodiment, the EIF2C1 locus is measured using a primer set comprising a forward primer 5′-GTTCGGCTTTCACCAGTCT and a reverse primer 5′-CTCCATAGCTCTCCCACTC. In another embodiment, the primer set is utilized for real-time PCR and further includes a probe. In another embodiment, the probe comprises the sequence and reporters as follows: 5′-HEX-CGCCCTGCCATGTGGAAGAT-BHQ1.

In any of the embodiments herein, a compromised renal status can comprise renal damage, renal injury, a renal disease, a renal disorder, renal graft rejection, or being non-responsive to a treatment for renal damage, renal injury, renal disease, renal disorder, or renal graft rejection. In any of the embodiments herein, the assessment of renal status can comprise measuring the progression of a renal disease, a renal injury, a renal graft injury, or a renal graft rejection. In any of the embodiments herein, the assessment of renal status can comprise measuring treatment response in and individual who is suffering from a renal disease, a renal injury, a renal graft injury, or a renal graft rejection and is currently undergoing or has undergone treatment.

In any of the embodiments herein, cfDNA can be extracted from the urine. In any one of the embodiments herein, the method can further comprise measurement of other pathological and clinical data. In any one of the embodiments herein, the method can further comprise measuring the amount of creatinine. In any one of the embodiments herein, the method can further comprise measuring proteinuria or cGFR. In any one of the embodiments herein, the method can further comprise performing a renal biopsy.

In any one of the embodiments herein, the individual is an individual at risk for renal damage, renal injury, a renal disease, a renal disorder, renal graft injury, or renal graft rejection. In any one of the embodiments herein, the individual is diabetic. In any one of the embodiments herein, the individual suffers from hypertension. In any one of the embodiments herein, the individual is a recipient of an allograft renal transplant. In any one of the embodiments herein, the individual is under treatment for renal graft injury or renal graft rejection. In any one of the embodiments herein, the individual has suffered at least one acute rejection episode.

In another aspect, the invention provides a diagnostic assay kit, the kit comprising: reagents for determining the copy number of at least one autosomal chromosome from a sample; a primer set used for determining said copy number; and instructions for use of the assay. In one embodiment, the autosomal chromosome is Chromosome 1. In another embodiment, the primer set is a set capable of amplifying an amplicon of locus EIF2C1. In another embodiment, the primer set comprises the forward primer 5′-GTTCGGCTTTCACCAGTCT and the reverse primer 5′-CTCCATAGCTCTCCCACTC. In another embodiment, the primer addition contains a probe useful for d PCR. In one embodiment, the sequence of the probe comprises the sequence and reporters as follows: 5′-HEX-CGCCCTGCCATGTGGAAGAT-BHQ1.

In any of the embodiments herein, the kit further comprises reagents for extracting cfDNA from a sample. In any of the embodiments herein, the sample comprises urine. In any of the embodiments herein, the primer set is capable of being used for real-time PCR, quantitative PCR, or digital PCR. In any of the embodiments herein, the primer set additionally comprises a probe useful for digital PCR.

In another aspect, the invention provides a diagnostic assay kit, the kit comprising: reagents for determining the number of ALU repeats in a sample; a primer set used for determining said number of ALU repeats; and instructions for use of the assay. In one embodiment, the primer set is a set capable of amplifying an amplicon of the ALU locus. In another embodiment, the primer set comprises the forward primer 5′-GCCTGTAATCCCAGCTACTC-3′ and the reverse primer 5′-ATCTCGGCTCACTGCAAC-3′. In another embodiment, the primer addition contains a probe useful for digital PCR. In one embodiment, the sequence of the probe comprises the sequence and reporters as follows: 5′-HEXTCAAGCGATTCTCCTGCCTCAGC-BHQ-3′. In another embodiment, the kit additionally comprises a primer set capable of determining the copy number of an autosomal chromosome. In one embodiment the autosomal chromosome is Chromosome 1. In another embodiment, the primer set is a set capable of amplifying an amplicon of locus EIF2C1. In another embodiment, the primer set comprises the forward primer 5′-GTTCGGCTTTCACCAGTCT and the reverse primer 5′-CTCCATAGCTCTCCCACTC. In another embodiment, the primer addition contains a probe useful for digital PCR. In one embodiment, the sequence of the probe comprises the sequence and reporters as follows: 5′-HEX-CGCCCTGCCATGTGGAAGAT-BHQ1.

In any of the embodiments herein, the kit further comprises reagents for extracting cfDNA from a sample. In any of the embodiments herein, the sample comprises urine. In any of the embodiments herein, the primer set is capable of being used for real-time PCR, quantitative PCR, or digital PCR. In any of the embodiments herein, the primer set additionally comprises a probe useful for digital PCR.

### BRIEF DESCRIPTION OF FIGURES

FIG. 1 presents a correlation between the copy numbers of Chromosome 1 Chromosome Y in plasma samples from individuals who underwent a renal transplant, with or without an acute rejection (AR) episode.

FIG. 2 presents a correlation between the copy numbers of Chromosome 1 Chromosome Y in urine samples from individuals who underwent a renal transplant, with or without an acute rejection (AR) episode.

FIG. 3 presents a quality control determination. In order to assess the robustness of the Chromosome 1 copy number assay in urine samples, the copy number of chromosome 1 was determined in 9 samples from 3 patients in repeated runs. A tight correlation was observed in between duplicate runs.

FIG. 4 shows that amount of protein in the urine correlated well with the Chromosome 1 copy number.

FIG. 5 shows the correlation in between ALU repeats and Chromosome 1 copy number in urine samples.

FIG. 6 shows a schematic of study samples and methods used Example 4. The study evaluated significance of measurement of cell-free DNA in the urine and plasma of renal transplant patients. The study was conducted in three phases. In phase 1, significance of dd-cfDNA in the urine and plasma was assessed. In Phase 2, measurement of a locus of an autosomal chromosome (Chr1) was evaluated. In phase 3, quantification of total cfDNA load in the urine was analyzed for specific graft injury detection.

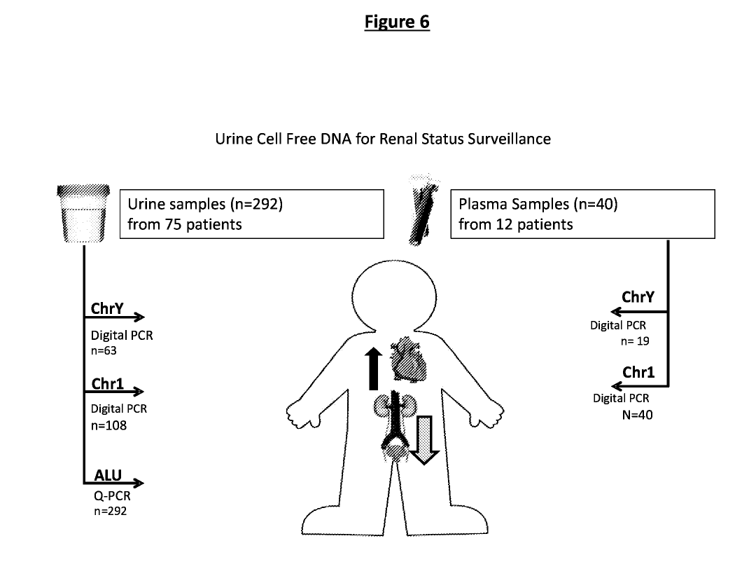

FIG. 7 shows that in a cohort of 19 female recipients with male grafts, an increase in quantities of plasma dd-cfDNA was observed at the time of acute rejection (AR) when compared to plasma dd-cfDNA values measured during stable (STA) graft function.

FIG. 8 shows that urine cfDNA load, as determined by the copy number of Chr 1 locus EIF2C1, demonstrated even the autosomal chromosomal DNA detected in the urine was donor derived. The urine cfDNA load for Chr Y locus SRY strongly correlated with the urine cfDNA load for Chr 1 locus EIF2C.

FIG. 9 shows cell-free Chr1 copy number in the urine as an indicator of renal transplant injury. (A) Copy number of locus EIF2C1 of Chr1 in the urine from renal transplant patients with transplant injury was significant when compared to copy number of locus EIF2C1 of Chr1 in the urine from renal transplant patients with no transplant injury. (B) The ROC analysis of the data resulted in an AUC of 0.777 with a p value <0.0001. (C) Protein to creatinine ratio in the urine of transplant injury phenotype was significantly higher when compared to protein to creatinine ratio of urine from patients with no transplant injury. (D) The ROC analysis resulted in an AUC of 0.66 with a p value 0.004.

FIG. 10 shows cell-free DNA for graft injury indication. (A) Data from a representative patient is shown when genome equivalents calculated from ALU assay data, there was an increased load of cfDNA in the urine of severe AR when compared to low grade AR or other chronic injuries. (B) The increase in total cfDNA in the urine was significantly higher in the urine of higher grade AR and reflux nephropathy compared to other chronic injuries in one representative case. (C) A sample data is presented when the increase in the urine cfDNA was particularly higher in AR compared to pre and post-AR samples (D) The increase in cfDNA load was higher in BKVN and the rise in cfDNA load could be observed in pre-BKVN urine.

FIG. 11 shows that a rise in the cfDNA load in the urine is increased in an AR episode when compared to pre- and post-AR. Three patients (patient #1, #2, and #3) were evaluated for total cfDNA in the urine pre and post-AR which demonstrated an elevation of cfDNA load in the urine during and AR episode.

### DETAILED DESCRIPTION

The present invention described herein provides, inter alia, compositions and methods that are useful for managing renal health (kidney health), renal disease, and kidney transplantation. The invention provide for diagnostic, prognostic, and/or therapeutic compositions and methods for assessing renal health of an individual (such as a patient), monitoring renal health and identifying individuals and sub-populations of individuals for therapeutic intervention. Generally, the invention is based on measuring cfDNA in urine. As detailed below, determining the copy number of autosomal chromosomes in urine cfDNA and/or determining the number of ALU repeats in cfDNA from the urine of those who have undergone an kidney/renal transplant, may be utilized to accurately identify individuals at risk for developing symptoms of renal graft injury or renal graft rejection, monitor the progression of renal graft injury or renal graft rejection, monitor the regression of renal graft injury or renal graft rejection, monitor the response to treatment in individuals being treated for renal graft injury or renal graft rejection, identify and/or predict the risk of the onset of renal graft injury or renal graft rejection, identify a sub-population of patients who should commence or continue treatment for renal graft injury or renal graft rejection, assess the efficacy of treatment for renal graft injury or renal graft rejection, and/or identify a sub-population of patients who should be monitored for developing symptoms of renal graft injury or renal graft rejection. Also as further detailed below, determining the copy number of autosomal chromosomes in urine cfDNA, and/or determining the number of ALU repeats in urine cfDNA may be utilized to accurately identify individuals at risk for developing renal disease or renal injury, monitor the progression of a renal disease or renal injury, monitor the regression of renal disease or renal injury, monitor the response to treatment in individuals being treated for renal disease or renal injury, identify and/or predict the risk of the onset of renal disease or renal injury, identify a sub-population of patients who should commence or continue treatment for renal disease or renal injury, assess the efficacy of treatment for renal disease or renal injury, and/or identify a sub-population of patients who should be monitored for developing symptoms of renal disease or renal injury symptoms.

### DEFINITIONS

For purposes of interpreting this specification, the following definitions will apply and whenever appropriate, terms used in the singular will also include the plural and vice versa.

As used herein, the terms “kidney” and “renal” are used interchangeably, the terms “renal health,” “kidney health,” “renal status,” and “kidney status” are used interchangeably, the terms “renal damage,” “kidney damage,” “renal injury,” and “kidney injury” are used interchangeably, the terms “renal disorder,” “kidney disorder,” “renal disease,” and “kidney disease” are used interchangeably, and the terms “renal graft” and “kidney graft” are used interchangeably.

The term “disorder” or “disease” and “injury” or “damage” are used interchangeably herein, refers to any alteration in the state of the body or one of its organs and/or tissues, interrupting or disturbing the performance of organ function and/or tissue function (e.g., causes organ dysfunction) and/or causing a symptom such as discomfort, dysfunction, distress, or even death to a subject afflicted with the disease.

An individual “at risk” of developing renal injury, renal disease or renal graft rejection may or may not have detectable disease or symptoms, and may or may not have displayed detectable disease or symptoms of disease prior to the treatment methods described herein. “At risk” denotes that an individual has one or more risk factors, which are measurable parameters that correlate with development of renal injury, renal disease, or renal graft rejection, as described herein and known in the art. A subject having one or more of these risk factors has a higher probability of developing renal injury, renal disease, or renal graft rejection than a subject without one or more of these risk factor(s).

An “individual” can be a “patient.” A “patient,” refers to an “individual” who is under the care of a treating physician. In one embodiment, the patient is suffering from renal damage or renal injury. In another embodiment, the patient is suffering from renal disease or disorder. In another embodiment, the patient has had a renal transplant and is undergoing of renal graft rejection. In yet other embodiments, the patient has been diagnosed with renal injury, renal disease, or renal graft rejection, but has not had any treatment to address the diagnosis.

A “patient sub-population,” and grammatical variations thereof, as used herein, refers to a patient subset characterized as having one or more distinctive measurable and/or identifiable characteristics that distinguishes the patient subset from others in the broader disease category to which it belongs. Such characteristics include having an elevated amount of urine cfDNA described herein as being characteristic of either having or being at risk for developing renal injury, renal disease, or renal graft rejection, optionally in combination with any of the symptoms described herein and known to one of skill in the art, including treating physicians.

The term “biological sample,” as used herein, refers to a composition that is obtained or derived from an individual that contains a cellular and/or other molecular entity that is to be characterized and/or identified, for example based on physical, biochemical, chemical and/or physiological characteristics. In one embodiment the sample is urine. In other embodiments, the sample is fluid (blood, serum, plasma, saliva, phlegm, gastric juices, semen, tears, sweat, etc.), skin, hair, cells, biopsies, or tissue specimens.

“Predicting” and “prediction” as used herein does not mean that the event will happen with 100% certainty. Instead it is intended to mean the event will more likely than not happen. Acts taken to “predict” or “make a prediction” can include the determination of the likelihood that an event will be more likely than not to happen. Assessment of multiple factors described herein can be used to make such a determination or prediction.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of autosomal copy number determination or ALU copy number evaluation performed on urine samples from an individual, one may use the results to determine whether a specific therapeutic regimen should be performed for that individual.

The term “diagnosis” is used herein to refer to the identification or classification of a medical or pathological state, disease or condition. For example, “diagnosis” may refer to identification of renal injury, renal disease, or renal graft rejection. “Diagnosis” may also refer to the classification of a severity of the renal injury, renal disease, or renal graft rejection. Diagnosis of the renal injury, renal disease, or renal graft rejection may be made according to any protocol that one of skill of art (e.g., a nephrologist) would use.

The term “aiding diagnosis” is used herein to refer to methods that assist in making a clinical determination regarding the presence, degree or other nature, of a particular type of symptom or condition of renal injury, renal disease, or renal graft rejection. For example, a method of aiding diagnosis of renal injury, renal disease, or renal graft rejection can include measuring the amount of any autosomal chromosome, Chromosome 1, Chromosome Y, or ALU repeats in a urine sample from an individual.

The term “prognosis” is used herein to refer to the prediction of the likelihood of the development and/or recurrence of renal injury, renal disease, or renal graft rejection. The predictive methods of the invention can be used clinically to make treatment decisions by choosing the most appropriate treatment modalities for any particular patient. The predictive methods of the present invention are valuable tools in predicting if and/or aiding in the diagnosis as to whether a patient is likely to develop renal injury, renal disease, or renal graft rejection, have recurrence of renal injury, renal disease, or renal graft rejection, and/or worsening of renal injury, renal disease, or renal graft rejection symptoms.

“Treating” and “treatment” refers to clinical intervention in an attempt to alter the natural course of the individual and can be performed before, during, or after the course of clinical diagnosis or prognosis. Desirable effects of treatment include preventing the occurrence or recurrence of renal injury, renal disease, or renal graft rejection or a condition or symptom thereof, alleviating a condition or symptom of renal injury, renal disease, or renal graft rejection, diminishing any direct or indirect pathological consequences of renal injury, renal disease, or renal graft rejection, decreasing the rate of renal injury, renal disease, or renal graft rejection progression or severity, and/or ameliorating or palliating the renal injury, renal disease, or renal graft rejection. In some embodiments, methods and compositions of the invention are used on patient sub-populations identified to be at risk of developing renal injury, renal disease, or renal graft rejection. In some cases, the methods and compositions of the invention are useful in attempts to delay development of renal injury, renal disease, or renal graft rejection. Beneficial or desired clinical results are known or can be readily obtained by one skilled in the art. For example, beneficial or desired clinical results can include, but are not limited to, one or more of the following: monitoring of renal injury, detection of renal injury, identifying type of renal injury, helping renal transplant physicians to decide whether or not to send transplant patients to go for a biopsy and make decisions for the purposes of clinical management and therapeutic intervention.

“Prophylaxis,” “prophylactic treatment,” “or preventive treatment” refers to prevention of the occurrence of one or more symptoms and/or their underlying cause, for example, prevention of a disease or condition in a patient susceptible to developing a disease or condition (e.g., at a higher risk, as a result of genetic predisposition, environmental factors, predisposing diseases or disorders, or the like).

As used herein, “delaying” the progression or development of renal injury, renal disease, or renal graft rejection means to defer, hinder, slow, retard, stabilize, and/or postpone development of the same. This delay can be of varying lengths of time, depending on the history of the disorder and/or individual being treated. The compositions and methods described herein can help to determine which individuals or patients might have a delay of renal injury, renal disease, or renal graft rejection.

The term “effective amount” refers to the amount of a pharmaceutical formulation for the treatment of renal injury, renal disease, or renal graft rejection in a sufficient amount to render a desired treatment outcome. An effective amount may be comprised within one or more doses, i.e., a single dose or multiple doses may be required to achieve the desired treatment endpoint. The compositions and methods described herein can help to determine which individuals or patients can be or should be receiving an effective amount of a pharmaceutical formulation.

A “therapeutically effective amount” refers to an amount of a pharmaceutical formulation for the treatment of a renal injury, renal disease, or renal graft rejection sufficient to produce a desired therapeutic outcome (e.g., reduction of severity of a disease or condition). A “prophylactically effective amount” refers to an amount of a pharmaceutical formulation for the treatment of a renal injury, renal disease, or renal graft rejection sufficient to prevent or reduce severity of a future disease or condition when administered to an individual who is susceptible and/or who may develop a disease or condition.

“Predicting” or “prediction” is used herein to refer to the likelihood that an individual is likely to respond either favorably or unfavorably to a treatment regimen.

As used herein, “at the time of starting treatment” or “baseline” refers to the time period at or prior to the first exposure to the treatment.

As used herein, “based upon” includes assessing, determining, or measuring the individual's characteristics as described herein (and preferably selecting an individual suitable for receiving treatment). When a copy number of an autosomal chromosome or total ALU copy number is used as a basis for selection, assessing, measuring, or determining method of treatment and/or prevention as described herein, the marker is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); or (f) predicting likelihood of clinical benefits. As would be well understood by one in the art, an evaluation of an individual's health-related quality of life in a clinical setting is a clear indication that this parameter was used as a basis for initiating, continuing, and/or ceasing administration of the treatments described herein.

It is understood that aspect and embodiments of the invention described herein include “consisting” and/or “consisting essentially of” aspects and embodiments.

As used herein, the term “detect” refers to the quantitative measurement of undetectable, low, normal, or high concentrations of one or more biomarkers such as, for example, nucleic acids, DNA, RNA, genomic DNA, dd-cfDNA, cfDNA, urinary DNA, and the like.

As used herein, the terms “quantify” and “quantification” may be used interchangeably, and refer to a process of determining the quantity or abundance of a substance in a sample (e.g., an autosomal chromosome), whether relative or absolute.

Reference to “about” a value or parameter herein includes (and describes) embodiments that are directed to that value or parameter per se. For example, description referring to “about X” includes description of “X.” The term “about” is used to provide flexibility to a numerical range endpoint by providing that a given value may be “a little above” or “a little below” the endpoint without affecting the desired result. Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

As used in the specification and the appended claims, the singular forms “a,” “an” and “the” include plural referents unless the context clearly indicates otherwise.

“Optional” or “optionally” means that the subsequently described event or circumstance can or cannot occur, and that the description includes instances where the event or circumstance occurs and instances where it does not.

As is apparent to one skilled in the art, an individual assessed, selected for, and/or receiving treatment may be an individual in need of assessment, selection for and/or receiving treatment.

### General Techniques

Unless defined otherwise, technical and scientific terms used herein have the same meaning as commonly understood by one of ordinary skill in the art to which this invention belongs.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); The Immunoassay Handbook (D. Wild, ed., Stockton Press NY, 1994); Bioconjugate Techniques (Greg T. Hermanson, ed., Academic Press, 1996); and Methods of Immunological Analysis (R. Masseyeff, W. H. Albert, and N. A. Staines, eds., Weinheim: VCH Verlags gesellschaft mbH, 1993). The practice of the invention may also employ multiple approaches of measurement of ALU repeats as described in Aging Cell (2013 Feb. 25, Characterization of the role of distinct plasma cfDNA (cfDNA) species in age-associated inflammation and frailty; Jylhävä J, Nevalainen T, Marttila S, Jylhä M, Hervonen A, Hurme M.), and Clinical Biochemistry (44 (2011) 1074-1079).

### Renal Status

The invention provides for compositions and methods that can be used to assess renal status in an individual. Such assessment is helpful for diagnosing when an individual is in need of medical intervention, such as being given more medication to address the medical problem or having medication decreased (including cessation) where it is no longer medically necessary. Compositions and method of the invention can be used to determine when an individual has altered renal status, compromised renal status, renal disease, renal damage, renal injury, renal graft rejection or being non-responsive to treatment for these conditions.

As used herein, a compromised renal status includes but is not limited to renal damage, renal injury, a renal disease, a renal disorder, renal graft rejection, or being non-responsive to a treatment for renal damage, renal injury, renal disease, renal disorder, or renal graft rejection. For example, an individual with a compromised renal status may be an individual at risk for developing symptoms of a renal disease or injury, an individual whose renal disease or injury has regressed, an individual at risk for developing symptoms of renal graft injury or renal graft rejection, or an individual whose renal graft injury or renal graft rejection has regressed.

As used herein, an altered renal status includes but is not limited to a change in an individual's renal damage, renal injury, renal disease, renal disorder, renal graft rejection, or responsiveness to a treatment for renal damage, renal injury, renal disease, renal disorder, or renal graft rejection.

Renal diseases or disorders are diverse, but individuals with renal disease frequently display characteristic clinical features. Common clinical conditions involving the kidney include but are not limited to the nephritic and nephrotic syndromes, renal cysts, acute kidney injury, chronic kidney disease, diabetes-induced nephropathy, urinary tract infection, nephrolithiasis, and urinary tract obstruction, glomerular nephritis (focal segmental glomerular sclerosis (FSGS), IgA nephropathy, mesangiocapillary, lupus and membranous etc), hypertensive nephropathy, and drug induced nephropathy. Renal diseases can also include the various cancers of the kidney which exist. For example such cancers include, but are not limited to, renal cell carcinoma, urothelial cell carcinoma of the renal pelvis, squamous cell carcinoma, juxtaglomerular cell tumor (reninoma), angiomyolipoma, renal oncocytoma, bellini duct carcinoma, clear-cell sarcoma of the kidney, mesoblastic nephroma, Wilms' tumor, mixed epithelial stromal tumors, clear cell adenocarcinoma, transitional cell carcinoma, inverted papilloma, renal lymphoma, teratoma, carcinosarcoma, and carcinoid tumor of the renal pelvis. Renal disease can also be virally induced and include, but are not limited to BKV nephropathy and nephropathy induced by EBV and CMV. Renal disease can also be drug-induced as some medications are nephrotoxic (they have an elevated risk for harming the kidneys). In the worst case, the drug causes kidney failure, while in other cases, the kidneys are damaged, but do not fail. Common nephrotoxic drugs include, but are not limited to, nonsteroidal anti-inflammatory drugs (NSAIDs), some antibiotics, some painkillers, and radiocontrast dyes used for some imaging procedures

Several renal conditions can be managed with removal of the kidney, or nephrectomy. When renal function, measured by glomerular filtration rate, is persistently poor, dialysis and kidney transplantation may be treatment options.

The indication for renal transplantation is end-stage renal disease (ESRD), regardless of the primary cause. Renal graft injury or renal graft rejection, renal allograft injury or renal allograft rejection can develop in patients who have undergone a renal transplant. This can happen because of several immune and non-immune factors such as ischemia reperfusion injury, size disparity, donor related factors, cell-mediated rejection, and antibody-mediated rejection, by way of example. Problems after a transplant may include: transplant rejection (hyperacute, acute or chronic), infections and sepsis due to the immunosuppressant drugs that are required to decrease risk of rejection, post-transplant lymphoproliferative disorder (a form of lymphoma due to the immune suppressants), imbalances in electrolytes including calcium and phosphate which can lead to bone problems among other things, and other side effects of medications including gastrointestinal inflammation and ulceration of the stomach and esophagus, hirsutism (excessive hair growth in a male-pattern distribution), hair loss, obesity, acne, diabetes mellitus type 2, hypercholesterolemia, and osteoporosis.

Individual and/or Patient Populations

The compositions and methods of the invention are applicable to any individual. In one embodiment, the individual is a patient. In other embodiments, the compositions and methods can be used to identify a sub-population of individuals (e.g., patients) who are in need of medical intervention.

In some embodiments, an individual whose urine cfDNA is examined to determine an autosomal chromosome copy number, Chromosome 1 copy number, or the number of ALU repeats, can be an individual who is at risk for developing a renal disease, renal injury, renal graft injury, or renal graft rejection, is suffering from a renal disease, renal injury, renal graft injury or renal graft rejection, is being treated for a renal disease, renal injury, renal graft injury, or renal graft rejection, whose renal disease, renal injury, renal graft injury, or renal graft rejection has progressed, or whose renal disease, renal injury, renal graft injury, or renal graft rejection has regressed. In other embodiments, an individual whose urine cfDNA is examined to determine an autosomal chromosome copy number, Chromosome 1 copy number, or the number of ALU repeats is an individual who has received a renal transplant, an allograft renal transplant, or has a family history of renal disease. In one embodiment, an individual whose urine cfDNA is examined to determine an autosomal chromosome copy number, Chromosome 1 copy number, or the number of ALU repeats, is an individual who has suffered an acute rejection (AR) episode following a kidney transplant. In yet other embodiments, an individual whose urine cfDNA is examined to determine an autosomal chromosome copy number, Chromosome 1 copy number, or the number of ALU repeats, is an individual who suffers from high blood pressure (hypertension), suffers from diabetes mellitus, suffers from systemic lupus erythematosus, or suffers from cardiovascular disease. In other embodiments, the individual is over 50 years of age, is over 55 years of age, is over 60 years of age, is over 65 years of age, is over 70 years of age, or is over 75 years of age.

### Methods of Use

Provided herein are methods of assessing renal status and evaluating renal health by examining urine cfDNA. Further provided herein are methods of assessing renal graft injury and renal graft rejection in kidney transplant patients.

Specifically provided herein are methods examine the cfDNA of individuals to identify individuals who are at risk for developing symptoms of renal graft injury or renal graft rejection, to monitor the progression of renal graft injury or renal graft rejection, to monitor the regression of renal graft injury or renal graft rejection, to monitor the response to treatment in individuals being treated for renal graft injury or renal graft rejection, to identify and/or predict the risk of the onset of renal graft injury or renal graft rejection, to identify a sub-population of patients who should commence or continue treatment for renal graft injury or renal graft rejection, to assess the efficacy of treatment for renal graft injury or renal graft rejection, and/or to identify a sub-population of patients who should be monitored for developing symptoms of renal graft injury or renal graft rejection. Also specifically provided herein are methods examine the cfDNA of individuals to identify individuals who are at risk for developing renal disease or renal injury, to monitor the progression of a renal disease or renal injury, to monitor the regression of renal disease or renal injury, to monitor the response to treatment in individuals being treated for renal disease or renal injury, to identify and/or predict the risk of the onset of renal disease or renal injury, to identify a sub-population of patients who should commence or continue treatment for renal disease or renal injury, to assess the efficacy of treatment for renal disease or renal injury, and/or to identify a sub-population of patients who should be monitored for developing symptoms of a renal disease or renal injury symptoms.

Such renal status assessment methods comprise measuring cfDNA extracted from a urine sample from an individual.

In one embodiment, the method to assess the renal status of an individual comprises determining the number of ALU repeats in a urine sample, and comparing the number of ALU repeats to either a standard number of ALU repeats in a urine sample from a normal population or to an otherwise pre-determined standard level, wherein a change in the number of ALU repeats is indicative of an altered renal status. If the copy number of Chromosome 1 is determined to be higher than the standard copy number, it is indicative of compromised renal status in the individual. If the copy number of Chromosome 1 is determined to be equal or lower than the standard copy number, it is indicative good renal health.

In another embodiment, the method to assess the renal status of an individual comprises determining the copy number of any chromosome in a urine sample, and comparing the copy number of the chromosome to either a standard copy number of that chromosome in a urine sample from a normal population or to an otherwise pre-determined standard level, wherein a change in the copy number is indicative of an altered renal status. If the copy number of the chromosome is determined to be higher than the standard copy number, it is indicative of compromised renal status in the individual. If the copy number of the chromosome is determined to be equal or lower than the standard copy number, it is indicative good renal health.

In another embodiment, the method to assess the renal status of an individual comprises determining the copy number of Chromosome 1, Chromosome 2, Chromosome 3, Chromosome 4, Chromosome 5, Chromosome 6, Chromosome 7, Chromosome 8, Chromosome 9, Chromosome 10, Chromosome 11, Chromosome 12, Chromosome 13, Chromosome 14, Chromosome 15, Chromosome 16, Chromosome 17, Chromosome 18, Chromosome 19, Chromosome 20, Chromosome 21, Chromosome 22, Chromosome X, and/or Chromosome Y in a urine sample, and comparing the copy number of the chromosome to either a standard copy number of that chromosome in a urine sample from a normal population or to an otherwise pre-determined standard level, wherein a change in the copy number is indicative of an altered renal status. If the copy number of the chromosome is determined to be higher than the standard copy number, it is indicative of compromised renal status in the individual. If the copy number of the chromosome is determined to be equal or lower than the standard copy number, it is indicative good renal health.

In another embodiment, the method to assess the renal status of an individual comprises determining the copy number of any autosomal chromosome in a urine sample, and comparing the copy number of the chromosome to either a standard copy number of that chromosome in a urine sample from a normal population or to an otherwise pre-determined standard level, wherein a change in the copy number is indicative of an altered renal status. If the copy number of the autosomal chromosome is determined to be higher than the standard copy number, it is indicative of compromised renal status in the individual. If the copy number of the autosomal chromosome is determined to be equal or lower than the standard copy number, it is indicative good renal health.

In another embodiment, the method to assess the renal status of an individual comprises determining the copy number of any sex chromosome in a urine sample, and comparing the copy number of the chromosome to either a standard copy number of that chromosome in a urine sample from a normal population or to an otherwise pre-determined standard level, wherein a change in the copy number is indicative of an altered renal status.

In another embodiment, the method to assess the renal status of an individual comprises determining the copy number of Chromosome 1 in a urine sample, and comparing the copy number of Chromosome 1 to either a standard copy number of Chromosome 1 in a urine sample from a normal population or to an otherwise pre-determined standard level, wherein a change in the copy number of Chromosome 1 is indicative of an altered renal status. If the copy number of Chromosome 1 is determined to be higher than the standard copy number, it is indicative of compromised renal status in the individual. If the copy number of Chromosome 1 is determined to be equal or lower than the standard copy number, it is indicative good renal health.

### Methods for Quantifying Cell Free, Determining Chromosomal Copy Number, and Number of ALU Repeats

In one embodiment cfDNA can be quantified from the urine of an individual. Urine can be collected by any standard method practiced by those skilled in the art. For example, urine can be obtained using the clean catch method. The clean catch method is one method of urine collection wherein the patient wipes genitalia with sanitation wipes, allows the first portion of urine go by, and subsequently collects the midstream urine in a sterile cup during urination. Using this method, 50-100 mL urine is collected and then centrifuged at 2000×g for 20 minutes. Circulating cfDNA from 5 mL of the supernatant is purified and concentrated using QIAamp Circulating Nucleic Acid Kit (Qiagen) following manufacturer's instructions.

In another embodiment circulating cfDNA can be quantified from the plasma of an individual. Plasma can be collected by any standard method practiced by those skilled in the art. In such an embodiment, the cfDNA can be purified and concentrated using a QIAamp Circulating Nucleic Acid Kit (Qiagen) or a similar product. Total cfDNA obtained either from urine or plasma can then quantified. One method to quantify total cfDNA is by using Quant-iT™ PicoGreen dsDNA Reagents and Kits (Invitrogen), following the manufacturer's instructions and protocols provided in Clinica Chimica Acta (327 (2003) 95-101 by Chen et al, (2003)). In one embodiment total cfDNA from a urine sample is quantified. In another embodiment, total cfDNA from the plasma is quantified.

In another embodiment the copy numbers of chromosomes or numbers of ALU repeats are be determined. In certain embodiments, in order to determine such copy numbers and ALU repeats, real-time PCR, quantitative PCR, and/or digital PCR are used. In other embodiments, methods such as fluorescent in situ hybridization, comparative genomic hybridization, and high-resolution array-based tests based on array comparative genomic hybridization (aCGH) and SNP array technologies are used.

In one embodiment, digital PCR can be used to determine the copy number of any chromosome, or the copy number of any autosomal chromosome, or the copy number of any sex chromosome. More specifically digital PCR can be used to determine the copy number of Chromosome 1, Chromosome 2, Chromosome 3, Chromosome 4, Chromosome 5, Chromosome 6, Chromosome 7, Chromosome 8, Chromosome 9, Chromosome 10, Chromosome 11, Chromosome 12, Chromosome 13, Chromosome 14, Chromosome 15, Chromosome 16, Chromosome 17, Chromosome 18, Chromosome 19, Chromosome 20, Chromosome 21, and/or Chromosome 22. Similarly digital PCR can be used to determine the copy number of Chromosome Y or Chromosome X. In one specific embodiment, digital PCR can be used to determine the copy number of Chromosome 1. In another general embodiment, digital PCR can be used to determine the number of ALU repeats in the sample.

In one embodiment a primer set capable of carrying out digital PCR, real-time PCR, or quantitative PCR amplifies an amplicon of at least 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 190, 200, 210, 220, 230, 250, or 300 base pairs. In another embodiment a primer set capable of carrying out digital PCR, real-time PCR, or quantitative PCR amplifies an amplicon of no more than 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 190, 200, 210, 220, 230, 250, or 300 base pairs. In any of the embodiments herein, the amplicon can be a range selected from any of the lower limits and upper limits above and described herein. In one specific embodiment, the amplicon is 81 base pairs in length. In another specific embodiment, the amplicon is 84 base pairs in length. In another specific embodiment, the amplicon is 115 base pairs in length.

In one embodiment a forward primer, a reverse primer, or probe capable of carrying out digital PCR, real-time PCR, or quantitative PCR is GC rich. In one embodiment a forward primer, a reverse primer, or probe capable of carrying out digital PCR, real-time PCR, or quantitative PCR is not GC rich. In another embodiment a primer set capable of carrying out digital PCR, real-time PCR, or quantitative PCR is at least 10%, 15%, 20%, 25%, 30%, 35%, 40%, 45%, 50%, 55%, 60%, 65%, 70%, 75%, or 80% GC rich. In another embodiment a primer set capable of carrying out digital PCR, real-time PCR, or quantitative PCR is no more than 10%, 15%, 20%, 25%, 30%, 35%, 40%, 45%, 50%, 55%, 60%, 65%, 70%, 75%, or 80% GC rich.

In one embodiment a forward primer, a reverse primer, or probe capable of carrying out digital PCR, real-time PCR, or quantitative PCR is at least 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, or 60 base pairs long. In one embodiment a forward primer, a reverse primer, or probe capable of carrying out digital PCR, real-time PCR, or quantitative PCR is no more than 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, or 60 base pairs long.

In one specific embodiment, digital PCR can be used to determine the number of ALU repeats. To carry out such an embodiment, a FastStart TaqMan Probe Master Mix with Rox (Roche) is prepared with the primers and probes appropriate for amplifying an amplicon of a locus of interest. In one embodiment, to amplify an ALU locus, the following primer set can be used: forward primer 5′-GGAGGCTGAGGCAGGAGAA-3′; reverse Primer 5′-ATCTCGGCTCACTGCAACCT-3′, and probe 5′-(FAM)CGCCTCCCGGGTTCAAGCG-3′. In another embodiment, to amplify of an 115 base pair ALU locus, the following primer set can be used: forward primer 5′-GCCTGTAATCCCAGCTACTC-3′; reverse primer 5′-ATCTCGGCTCACTGCAAC-3′; and probe 5′-HEXTCAAGCGATTCTCCTGCCTCAGC-BHQ-3′.

In another specific embodiment, digital PCR can be used to determine the copy number of Chromosome 1. To carry out such an embodiment, a FastStart TaqMan Probe Master Mix with Rox (Roche) is prepared with the primers and probes appropriate for amplifying an amplicon of a locus of interest. For example, to amplify an 81 base pair amplicon of locus EIF2C1 on Chromosome 1, the following can be used: forward primer 5′-GTTCGGCTTTCACCAGTCT; reverse primer 5′-CTCCATAGCTCTCCCACTC; probe HEX-CGCCCTGCCATGTGGAAGAT-BHQ1.

In another specific embodiment, digital PCR can be used to determine the copy number of Chromosome Y. To carry out such an embodiment, a FastStart TaqMan Probe Master Mix with Rox (Roche) is prepared with the primers and probes appropriate for amplifying an amplicon of a locus of interest. For example, to amplify an 84 base pair amplicon of locus DYS 14 on Chromosome Y (a multi-copy locus), the following primer set can be used: forward primer 5′-ATCGTCCATTTCCAGAATCA; reverse primer 5′-GTTGACAGCCGTGGAATC; probe: 5′-FAM-TGCCACAGACTGAACTGAATGATTTTC-BHQ1

In another specific embodiment, digital PCR can be used to determine the copy number of Chromosome Y. To carry out such an embodiment, a FastStart TaqMan Probe Master Mix with Rox (Roche) is prepared with the primers and probes appropriate for amplifying an amplicon of a locus of interest. For example, to amplify an 84 base pair amplicon of locus SRY on Chromosome Y (a single-copy locus), the following primer set can be used: forward primer 5′-CGCTTAACATAGCAGAAGCA; reverse primer 5′-AGTTTCGAACTCTGGCACCT; and probe 5′-FAM-TGTCGCACTCTCCTTGTTTTTGACA-BHQ1.

The samples prepared for digital PCR can be loaded onto 12.765 digital array chips using the BioMark real-time PCR system (Fluidigm). Data can be extracted and estimated using Digital PCR Analysis Software v3.0. The number of copies of each chromosome can be calculated using the following equation: (382/panel/4.59 μl/panel)×(8 μl reaction mix/1 μl DNA)=Copies/μl in original sample.

Correlation between the copy numbers of one chromosome and another chromosome, between one locus on one chromosome and another locus on the same chromosome, or between the copy number of one chromosome and the number of ALU repeats can be quantified and expressed. Such correlations can be carried out to determine the sensitivity and predictability of particular primer sets to detect renal disease, renal injury, renal graft injury, or renal graft rejection. Such correlations can be also carried out in order to optimize the primer sets for particular body fluids, or samples.

As described herein, the quantification and determination of the total cfDNA, of the copy number of at least one chromosome, of the copy number of at least one autosomal chromosome, of the copy number of Chromosome 1, of the copy number of at least one sex chromosome, or of the quantification of the number of ALU repeats can be carried out using urine and/or plasma samples, and combined with a measurement of urine protein. In one specific embodiment the urine protein is creatinine. Furthermore, the quantification and determination of the total cfDNA, of the copy number of at least one chromosome, of the copy number of at least one autosomal chromosome, of the copy number of Chromosome 1, of the copy number of at least one sex chromosome, or of the quantification of the number of ALU repeats can be carried out using urine and/or plasma samples, and combined with measurement of other pathological and clinical data. Such pathological and clinical data include but are not limited to medical imaging, other blood tests, renal biopsies, glomerual filtration rate (GFR) measurements (the amount of plasma water that is filtered per minute), corrected GFR (cGFR) measurements GFR with a correction factor for body surface area), proteinuria measurements, and the like.

### Standard Levels

The methods as provided herein are used to assess the renal status of an individual by, in part, determining the copy number of at least one chromosome, determining the copy number of at least one autosomal chromosome, determining the copy number of at least sex chromosome, determining the copy number of Chromosome 1 in particular, determining the copy number of Chromosome Y in particular, determining the number of ALU repeats in a urine sample, and comparing that number to either a standard copy number in a urine sample from a normal population or to an otherwise pre-determined standard level, wherein a change in the number is indicative of an altered renal status.

In one embodiment a standard copy number of a chromosome in a urine sample from a normal population is determined by measuring the mean number of copies of that particular chromosome in a normal population. In one embodiment a standard copy number of a chromosome in a urine sample from a normal population is determined by measuring the median number of copies of that particular chromosome in a normal population. In one embodiment a standard copy number of a chromosome in a urine sample from a normal population is determined by measuring the range of the number of copies of that particular chromosome in a normal population.

In one embodiment a standard copy number for Chromosome 1 in a urine sample from a normal population is determined by measuring the mean number of copies of Chromosome 1 in a normal population. In one embodiment a standard copy number for Chromosome 1 in a urine sample from a normal population is determined by measuring the median number of copies of Chromosome 1 in a normal population. In one embodiment a standard copy number for Chromosome 1 in a urine sample from a normal population is determined by measuring the range of the number of copies of Chromosome 1 in a normal population.

In one embodiment a standard number of ALU repeats in a urine sample from a normal population is determined by measuring the mean number of repeats of the ALU locus in a normal population. In one embodiment a standard copy number for Chromosome 1 in a urine sample from a normal population is determined by measuring the median number of repeats of the ALU locus in a normal population. In one embodiment a standard number of ALU repeats in a urine sample from a normal population is determined by measuring the range of the number of ALU repeats in a normal population.

In one embodiment a normal population is a population of individuals who are not suffering or have never suffered from renal injury, renal disease, renal transplant injury, or renal transplant rejection. In another embodiment, a normal population is a population of individuals whose copy numbers were determined prior to suffering renal injury, renal disease, renal transplant injury, or renal transplant rejection. In another embodiment a normal population is a population of individuals who suffered from renal injury, renal disease, renal transplant injury, or renal transplant rejection but then recovered. In various embodiments a normal population is a population of individuals who are age matched, sex matched, matched for disease history (such as diabetes, cardiovascular disease, hypertension, prior viral infection, renal cancers, or the like), matched for blood type, matched for racial origin, matched for ethnicity, matched for transplant history, matched for any other variable that could lead to heterogeneity in determining values for a normal population, and combinations thereof.

In one embodiment, the number as determined from the sample is equal to or substantially the same as the copy number in a urine sample from a normal population or an otherwise pre-determined standard level. In another embodiment, the number as determined from the sample is less than the copy number in a urine sample from a normal population or an otherwise pre-determined standard level. In another embodiment, the number as determined from the sample is greater than the copy number in a urine sample from a normal population or an otherwise pre-determined standard level. In a related embodiment, the number as determined from the sample is at least 2, at least 3, at least 4, at least 5, at least 6, at least 7, at least 8, at least 9, at least 10, at least 15, at least 20, at least 25, at least 50, at least 75, at least 100, at least 250, at least 500, at least 750, at least 1000, at least 2500, at least 5000, at least 7500, at least 10,000, at least 50,000, at least 100,000, at least 500,000, at least 1,000,000, at least 10,000,000, or at least even 100,000,000 times greater than the copy number in a urine sample from a normal population or an otherwise pre-determined standard level. In another embodiment, the number as determined from the sample is less than the copy number in a urine sample from a normal population or an otherwise pre-determined standard level.

### Kits

The present invention also provides for kits comprising materials useful for carrying out the diagnostic and prognostic methods of the invention. The procedures described herein may be performed by clinical laboratories, experimental laboratories, or practitioners. The invention provides kits which can be used in these different settings.

In one embodiment, an inventive kit comprises at least one primer set used to quantify the copy number of at least one chromosome, as described herein. In certain embodiments, the kit comprises at least one primer set used to quantify the copy number of any chromosome, or more specifically the copy number of any autosomal chromosome, the copy number of Chromosome 1, the copy number of Chromosome Y, or the number of ALU repeats present in a sample obtained from an individual. In one embodiment, the sample is urine obtained from the individual. The primer set is provided, preferably, in an amount that is suitable for detecting cfDNA in a urine sample.

In one embodiment, an inventive kit comprises a primer set used to quantify the number of copies of any autosomal chromosome in the cfDNA from a urine sample. In another embodiment, the primer set is designed to target a locus of the chromosome that is relatively constant across the population. In another embodiment, the primer set is designed to target a locus of the chromosome, or a portion of the chromosome that represents a constant region. In another embodiment, the primer set is designed to target a non-variable region, or a non-hypervariable region of the chromosome. In another embodiment, the primer set is designed to target a locus of the chromosome that is not constant across the population. In another embodiment, the primer set is designed to target a locus of the chromosome, or a portion of the chromosome that represents a variable region. In another embodiment, the primer set is designed to target a hypervariable region of the chromosome. One of the skill in the art, using the genomic methods and databases currently available, will be able to identify those regions of a chromosome or gene locus that are constant, not constant, variable, non-variable, hypervariable, non-hypervariable, and the like.

In certain embodiments, an inventive kit comprises a primer set used to quantify the number of ALU repeats in the cfDNA extracted from a urine sample obtained from an individual, as described herein.

In one embodiment the primer set is capable of amplifying a 115 base pair amplicon of the ALU locus. In this embodiment the primer set can comprise the forward primer 5′-GCCTGTAATCCCAGCTACTC-3′ and the reverse primer 5′-ATCTCGGCTCACTGCAAC-3′. In one specific embodiment this primer set is capable of being used for digital PCR and the primer set additionally comprises a probe useful for digital PCR. One exemplary sequence for such a probe is 5′-HEXTCAAGCGATTCTCCTGCCTCAGC-BHQ-3′.

In another embodiment the primer set is capable of amplifying another amplicon of an ALU locus. In this embodiment the primer set can comprise the forward primer 5′-GGAGGCTGAGGCAGGAGAA-3′ and the reverse primer 5′-ATCTCGGCTCACTGCAACCT-3′. In one specific embodiment this primer set is capable of being used for digital PCR and the primer set additionally comprises a probe useful for digital PCR. One exemplary sequence for such a probe is 5′(FAM)CGCCTCCCGGGTTCAAGCG-3′.

In certain embodiments, an inventive kit comprises a primer set used to quantify the number of copies of Chromosome 1, Chromosome 2, Chromosome 3, Chromosome 4, Chromosome 5, Chromosome 6, Chromosome 7, Chromosome 8, Chromosome 9, Chromosome 10, Chromosome 11, Chromosome 12, Chromosome 13, Chromosome 14, Chromosome 15, Chromosome 16, Chromosome 17, Chromosome 18, Chromosome 19, Chromosome 20, Chromosome 21, and/or Chromosome 22 in the cfDNA extracted from a urine sample obtained from an individual, as described herein.

In certain embodiments, an inventive kit comprises a primer set used to quantify the number of copies of Chromosome 1 in the cfDNA extracted from a urine sample obtained from an individual, as described herein. In one embodiment the primer set is capable of amplifying an amplicon of locus EIF2C1. In this embodiment the primer set can comprise the forward primer 5′-GTTCGGCTTTCACCAGTCT and the reverse primer 5′-CTCCATAGCTCTCCCACTC. In one embodiment the primer set is capable of being used for digital PCR and the primer set additionally comprises a probe useful for digital PCR. One exemplary sequence for such a probe is 5′-HEX-CGCCCTGCCATGTGGAAGAT-BHQ1.

In certain embodiments, an inventive kit comprises a primer set used to quantify the number of copies of Chromosome Y in the cfDNA extracted from a urine sample obtained from an individual, as described herein. In one embodiment the primer set is capable of amplifying an amplicon of locus DYS 14. In this embodiment the primer set can comprise the forward primer 5′-ATCGTCCATTTCCAGAATCA and the reverse primer 5′-gttgacagccgtggaatc. In one embodiment the primer set is capable of being used for digital PCR and the primer set additionally comprises a probe useful for digital PCR. One exemplary sequence for such a probe is 5′-FAM-TGCCACAGACTGAACTGAATGATTTTC-BHQ1. In another embodiment the primer set is capable of amplifying an amplicon of locus SRY. In this embodiment the primer set can comprise the forward primer 5′-CGCTTAACATAGCAGAAGCA and the reverse primer 5′-AGTTTCGAACTCTGGCACCT. In one embodiment the primer set is capable of being used for digital PCR and the primer set additionally comprises a probe useful for digital PCR. One exemplary sequence for such a probe is 5′-FAM-TGTCGCACTCTCCTTGTTTTTGACA-BHQ1.

In certain embodiments, an inventive kit comprises a primer set used to quantify the number of copies of Chromosome X in the cfDNA extracted from a urine sample obtained from an individual, as described herein.

In certain embodiments, an inventive kit comprises one or more primers that may be immobilized on a substrate surface (e.g., beads, array and the like).

As amenable, these suggested kits components may be packaged in a manner customary for use by those of skill in the art. For example, these suggested kit components may be provided in solution or as a liquid dispersion or the like. The different reagents included in an inventive kit may be supplied in a solid (e.g., lyophilized) or liquid form. The kits of the present invention may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the disclosed methods may also be provided. The individual containers of the kit are preferably maintained in close confinement for commercial sale.

In certain embodiments, a kit further comprises instructions for using its components for the diagnosis of renal status, renal transplant status, renal disease, renal injury, or renal graft rejection in an individual according to a method of the invention. Instructions for using the kit according to methods of the invention may comprise instructions for processing the biological sample obtained for the individual and/or for performing the test, and/or instructions for interpreting the results.

A kit may also contain a notice in the form prescribed by a governmental agency regulating the manufacture, use or sale of pharmaceuticals or biological products.

### Computer Programs

Any of the methods above can be performed by a computer program product that comprises a computer executable logic that is recorded on a computer readable medium. For example, the computer program can execute some or all of the following functions: (i) controlling isolation of nucleic acids from a sample, (ii) pre-amplifying nucleic acids from the sample, (iii) amplifying specific regions in the sample, (iv) identifying and quantifying total cfDNA, a chromosomal copy number, or number of ALU repeats in the sample, (v) comparing data as detected from the sample with a reference standard, (vi) determining a renal status or clinical outcome, (vi) declaring normal or abnormal renal status or clinical outcome.

The computer executable logic can work in any computer that may be any of a variety of types of general-purpose computers such as a personal computer, network server, workstation, or other computer platform now or later developed. In some embodiments, a computer program product is described comprising a computer usable medium having the computer executable logic (computer software program, including program code) stored therein. The computer executable logic can be executed by a processor, causing the processor to perform functions described herein. In other embodiments, some functions are implemented primarily in hardware using, for example, a hardware state machine. Implementation of the hardware state machine so as to perform the functions described herein will be apparent to those skilled in the relevant arts.

The program can provide a method of evaluating a renal status or clinical outcome in a individual at risk for developing, or suffering from renal disease, renal injury, renal graft injury, or renal graft rejection.

### EXAMPLES

### Example 1

### Detection of Cell Free DNA in Plasma by Determining Chromosome Y and Chromosome 1 Copy Numbers

Chromosome Y and Chromosome 1 copy numbers were quantified in 33 unique plasma samples from 10 renal transplant patients with or without acute rejection (AR) episodes. 3 patients were female recipients of a male kidney. The other 7 patients had other gender combinations. 9 out of 10 patients had biopsy proven injuries. For each patient, 2-4 serial plasma samples were analyzed.

Circulating cfDNA from 3 mL of plasma was purified and concentrated using QIAamp Circulating Nucleic Acid Kit (Qiagen) following manufacturer's instructions. Total DNA was then quantified using Quant-iT™ PicoGreen dsDNA Reagents and Kits (Invitrogen) following manufacturer's instructions.

Digital PCR was used to determine the copy numbers of Chromosome Y and Chromosome 1. The FastStart TaqMan Probe Master Mix with Rox (Roche) was prepared with the following primers and probes. To amplify an 84 base pair amplicon of locus DYS 14 on Chromosome Y (multi-copy locus), the following were used: forward primer 5′-ATCGTCCATTTCCAGAATCA; reverse primer 5′-gttgacagccgtggaatc; and probe 5′-FAM-TGCCACAGACTGAACTGAATGATTTTC-BHQ1.

To amplify an 81 base pair amplicon of locus EIF2C1 on Chromosome 1, the following were used: forward primer 5′-GTTCGGCTTTCACCAGTCT; reverse primer 5′-CTCCATAGCTCTCCCACTC; probe HEX-CGCCCTGCCATGTGGAAGAT-BHQ1.

The samples were loaded onto 12.765 digital array chips using the BioMark real-time PCR system (Fluidigm). Total DNA content of the samples was determined using PicoGreen to ensure equal loading. Control male and female genomic DNA (Promega) was used to calibrate the Chromosome 1 and Chromosome Y signals.

Data were extracted and estimated using Digital PCR Analysis Software v3.0. The number of copies of each chromosome were calculated using the following equation: (382/panel/4.59 μl/panel)×(8 μl reaction mix/1 μl DNA)=Copies/μl in original sample.

In order to assess if the copy number of Chromosome 1 in plasma samples correlates with the copy number of Chromosome Y, 13 plasma samples from 4 patients female recipients of a male kidney were analyzed (of which 3 patients had biopsy proven graft injury).

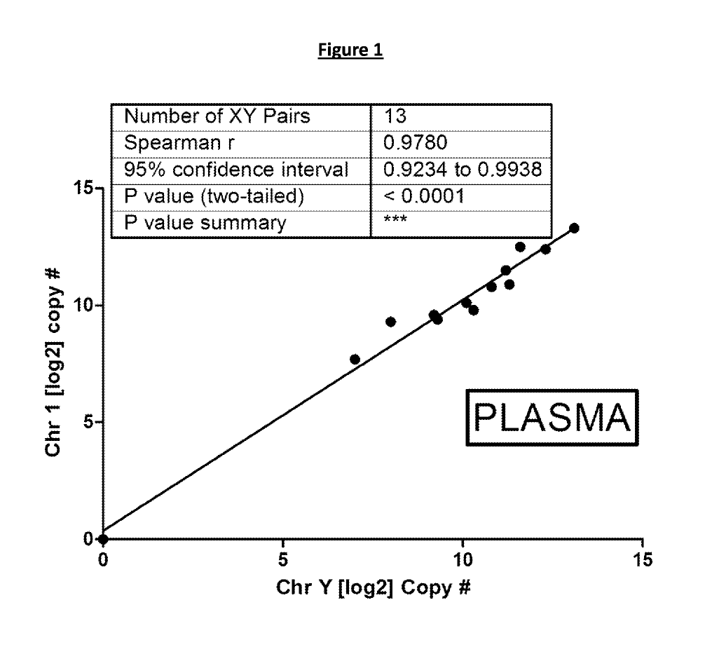

FIG. 1 presents a correlation between the copy numbers of Chromosome 1 Chromosome Y in plasma samples from individuals who underwent a renal transplant, with or without an acute rejection (AR) episode. In plasma, Chromosome Y was detected, with the use of DYS-14 primers directed at a multi-locus gene. The sensitivity of the cell-free copy number of Chromosome 1 as a measure for detecting kidney injury by measuring cfDNA from plasma was only 33% as sensitive when compared to measuring cfDNA from urine.

These data showed that plasma is a less than optimal body fluid to measure cfDNA to monitor kidney injury.

### Example 2

### Detection of Cell Free DNA in Urine by Determining Chromosome Y and Chromosome 1 Copy Numbers

Chromosome Y and Chromosome 1 copy numbers were quantified in 125 unique urine samples from 40 renal transplant patients with or without acute rejection (AR) episodes. Of the 125 samples, 20 were taken from an individual who had had an AR episode or were at the borderline of an AR episode and 105 samples were taken from individuals who had not had an AR episode. 53 of the 125 urine samples were from 16 female recipients of a male kidney. The remaining 72 urine samples were from 24 patients with other gender combinations. For each patient, 2-4 serial samples were analyzed. 50-100 mL urine was obtained by the clean catch method. The clean catch method is one method of urine collection wherein the patient wipes genitalia with sanitation wipes, allows the first portion of urine go by, and subsequently collects the midstream urine in a sterile cup during urination. The samples were centrifuged at 2000×g for 20 minutes. Circulating cfDNA from 5 mL of the supernatant was purified and concentrated using QIAamp Circulating Nucleic Acid Kit (Qiagen) following manufacturer's instructions. Total DNA was then quantified using Quant-iT™ PicoGreen dsDNA Reagents and Kits (Invitrogen) following manufacturer's instructions.

The copy numbers of Chromosome Y and Chromosome 1 were determined using the BioMark (Fluidigm) real-time PCR system. The FastStart TaqMan Probe Master Mix with Rox (Roche) was prepared with the following primers and probes. To amplify an 84 base pair amplicon of locus SRY on Chromosome Y (single-copy locus), the following were used: forward primer 5′CGCTTAACATAGCAGAAGCA; reverse primer 5′-AGTTTCGAACTCTGGCACCT; probe 5′FAM-TGTCGCACTCTCCTTGTTTTTGACA-BHQ1. To amplify an 81 base pair amplicon of locus EIF2C1 on Chromosome 1, the following were used: forward primer 5′-GTTCGGCTTTCACCAGTCT; reverse primer 5′-CTCCATAGCTCTCCCACTC; probe HEX-CGCCCTGCCATGTGGAAGAT-BHQ1. The samples were then loaded onto 12.765 digital array chips (Fluidigm). Total DNA content of the samples were determined using PicoGreen, as described above, and was used to ensure equal loading. Control male and female genomic DNA (Promega) was used to calibrate the Chromosome 1 and Chromosome Y signals.

Data were extracted and estimated using Digital PCR Analysis Software v3.0. The number of copies of each chromosome were calculated using the following equation: (382/panel/4.59 μl/panel)×(8 μl reaction mix/1 μl DNA)=Copies/μl in original sample.

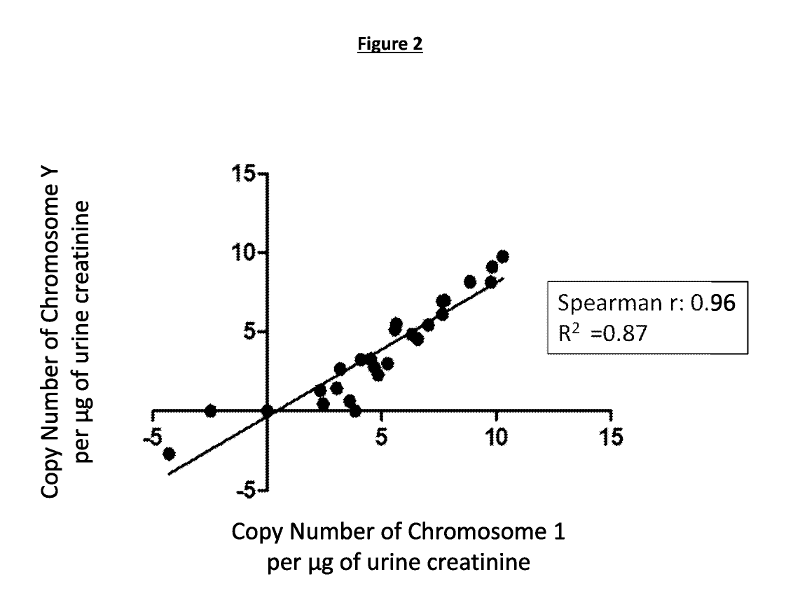

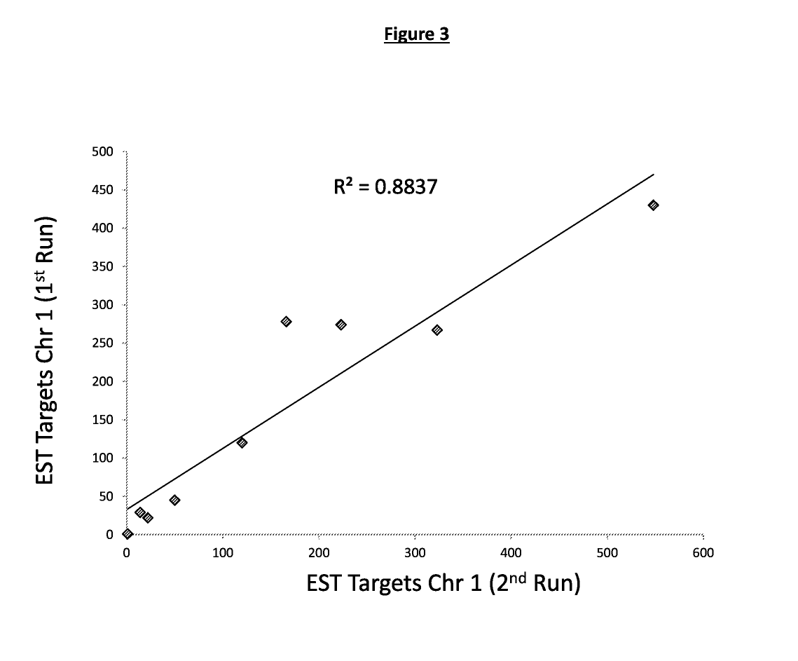

In order to assess if the copy number of Chromosome 1 in the urine samples correlated with the copy number of Chromosome Y, their respective numbers were compared in FIG. 2. FIG. 2 presents a correlation between the copy numbers of Chromosome 1 Chromosome Y in 27 urine samples from 12 female individuals who underwent a renal transplant and received a male kidney, with or without an acute rejection (AR) episode. As a quality control and in order to assess the robustness of the Chromosome 1 copy number assay in urine samples, the copy number of Chromosome 1 was determined in 9 samples from 3 patients in repeated runs. A tight correlation was observed in between duplicate runs as is presented in Table 1 and FIG. 3. Table 1 shows a strong reproducibility in between two runs in terms of estimated targets. ‘Estimated targets’ a unitless number that is obtained from the Fluidigm Biomark instrument. Furthermore, no Chromosome Y was detected in samples where a female patient received a renal transplant from a female donor.

TABLE 1

| | Run 1 | Run 2 |
|---|---|---|
| | (Estimated Targets) | (Estimated Targets) |
| | Chromosome 1 | Chromosome 1 |
| Patient 1 | 323 | 267 |
| | 1 | 1 |
| | 22 | 22 |
| Patient 2 | 548 | 430 |
| | 223 | 274 |
| | 166 | 278 |
| Patient 3 | 14 | 29 |
| | 120 | 120 |
| | 50 | 45 |

The mean number of copies of Chromosome 1 per mL per urine in individuals who had received a transplant but exhibited no injury (non-transplant injury phenotype) was 1681±3124 (standard deviation). The median number of copies of Chromosome 1 per mL per urine in individuals who had received a transplant but exhibited no injury was 321. The range of copies of Chromosome 1 per mL per urine in individuals who had received a transplant but exhibited no injury was 0-12963 copies.

The mean number of copies of Chromosome 1 per mL per urine in individuals who had received a transplant and exhibited injury (injury phenotype) was 10728±30283 (standard deviation). The median number of copies of Chromosome 1 per mL per urine in individuals who had received a transplant but exhibited no injury was 2009. The range of copies of Chromosome 1 per mL per urine in individuals who had received a transplant but exhibited no injury was 0-198496 copies.

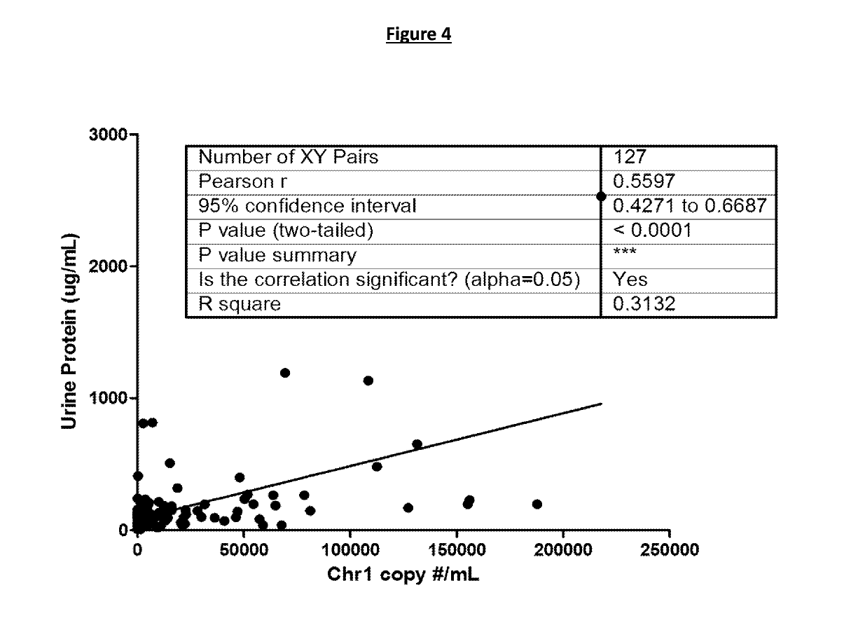

The current standard method to assess kidney status is to measure urine protein (to measure proteinuria). In order to evaluate the correlation of Chromosome 1 copy number with the measurement of urine protein, urine protein was compared to Chromosome 1 copy number. As shown in FIG. 4, the amount of urine protein correlated well with Chromosome 1 copy number as determined in the assay described above. Urine protein was measured by a standard Bradford assay (Coomassie Plus (Bradford) Protein Assay, Thermo Scientific) and urine creatinine was measured using QuantiChrom™ Creatinine Assay Kit (BioAssay Systems) following the manufacturer's protocol.

The urinary cell-free Chromosome 1 copy number correlated well with pathological and clinical data. These data are presented in Table 2. Chromosome 1 copy number correlates better with cGFR than proteinuria does with cGFR. Similarly, Chromosome 1 copy number correlates better with acute injury than proteinuria correlates with acute injury. The Chromosome 1 copy number/μg urine creatinine correlated with the acute injury score (i) with an r=0.32 which is better than how conventional proteinuria correlates with the acute injury score, r=0.22. Chromosome 1 copy number/μg urine creatinine correlated with cGFR measurements with r=−0.28, which is better than how conventional proteinuria correlates with cGFR, r=−0.02.

TABLE 2

| | cGFR (P) | cGFR (r) | I (P) | i(r) | T (P) | t(r) | G (P) | g (r) | CADI (P) | CADI (r) |
|---|---|---|---|---|---|---|---|---|---|---|
| Chromosome 1/ | 7.70E−03 | −0.28 | 1.20E−03 | 0.32 | 1.3E−03 | 0.32 | 2.30E−02 | 0.23 | 7.1E−02 | 0.18 |
| Creatine (log2) | | | | | | | | | | |

(P) = p-value,
(r) = correlation coefficient, interstitium (i), tubules (t), and glomeruli (g);
CADI = Chronic Allograft Damage Index (CADI)

### Example 3

### Detection of Cell Free DNA in Urine by Determining Number of ALU Repeats and the Correlation Between ALU Repeats and Chromosome 1 Copy Number

The number of ALU repeats was determined by standard real time PCR in the urine samples described in Example 2. ALU repeats and Chromosome 1 copy numbers were quantified in 125 unique urine samples from 40 renal transplant patients with or without acute rejection (AR) episodes. Of the 125 samples, 20 were taken from an individual who had had an AR episode or were at the borderline of an AR episode and 105 samples were taken from individuals who had not had an AR episode. For each patient, 2-4 serial samples were analyzed. cfDNA was isolated and purified, as above in Example 2. Total DNA was quantified using Quant-iT™ PicoGreen dsDNA Reagents and Kits (Invitrogen) following manufacturer's instructions.

The copy numbers of Chromosome Y and the number of ALU repeats were determined using the BioMark (Fluidigm) real-time PCR system. The FastStart TaqMan Probe Master Mix with Rox (Roche) was prepared with the following primers and probes. To amplify an 81 base pair amplicon of locus EIF2C1 on Chromosome 1, the following were used: forward primer 5′-GTTCGGCTTTCACCAGTCT; reverse primer 5′-CTCCATAGCTCTCCCACTC; probe HEX-CGCCCTGCCATGTGGAAGAT-BHQ1. To amplify an 115 base pair amplicon of an ALU locus, the following were used: forward primer 5′-GCCTGTAATCCCAGCTACTC; reverse primer 5′-ATCTCGGCTCACTGCAAC; probe 5′-HEX-TCAAGCGATTCTCCTGCCTCAGC-BHQ-3′. The samples were then loaded onto 12.765 digital array chips (Fluidigm). Total DNA content of the samples were determined using PicoGreen, as described above, and was used to ensure equal loading. Control male and female genomic DNA (Promega) was used to calibrate the Chromosome 1 and ALR repeat signals.

Data were extracted and estimated using Digital PCR Analysis Software v3.0. The number of copies of each chromosome was calculated using the following equation: (382/panel/4.59 μl/panel)×(8 μl reaction mix/1 μl DNA)=Copies/μl in original sample.

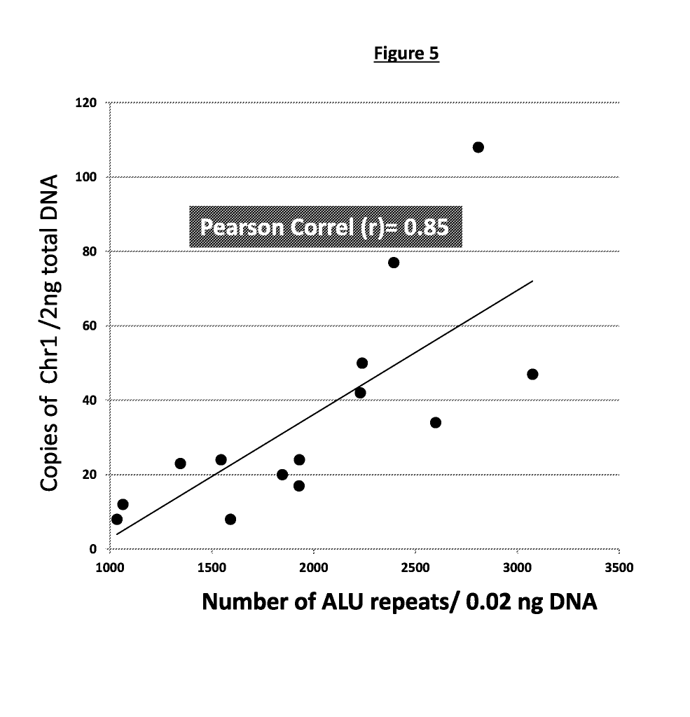

This example demonstrates the utility of using genomic autosomal DNA in assessing renal status and renal injury. FIG. 5 shows the correlation in between ALU repeats and Chromosome 1 copy number in samples. The number of Chromosome 1 copies per 2 ng of total DNA was plotted versus the number of ALU repeats per 0.02 ng of total DNA and showed a good correlation of Chromosome 1 number to number of ALU repeats.

### Example 4

### Large Scale Study

Patients and Samples: This study enrolled kidney transplant patient plasma and urine samples, collected contemporaneously with a renal allograft indicated or protocol biopsy from pediatric and young adult recipients of kidney transplants from 2004 to 2010 at Lucile Packard Children's Hospital at Stanford. Patients included in the study where those who had at least two samples during the study period with a matched transplant biopsy, which included all available surveillance and indication (allograft dysfunction) driven urine samples for each patient selected. The study was approved by the Ethics Committee of Stanford University Medical School and California Pacific Medical Center Research Institute (CPMCRI), and all patients/guardians provided informed consent to participate in the research, in full adherence to the Declaration of Helsinki 292 unique urine samples were collected from 75 kidney transplant patients, at surveillance post-transplant time points of 3, 6, 12, and 24 months post-transplant and at the time indication biopsies. Plasma samples (n=40) were collected from 12 patients at surveillance time points 3, 6, and 12 months post-transplant, and at indication. Deidentified clinical information was collected on all participating patients. All matched biopsies were blindly scored by a single pathologist using the most recent Banff criteria for both acute and chronic injury. Acute rejection (AR) was defined at minimum, as per Banff Schema, a tubulitis score ≧1 accompanied with an interstitial inflammation score ≧1. Chronic allograft injury (CAI) was defined at minimum, as tubular atrophy score ≧1 accompanied by an interstitial fibrosis score ≧1. BK virus nephritis (BKVN) was identified by presence of BK virus in the urine and the plasma and demonstration of inflammation and a positive SV40 stain in the allograft. Acute tubular necrosis (ATN) was also diagnosed. Pyelonephritis was defined as presence of pyurina and bacterial urinary tract infection and a positive blood culture for bacterial sepsis. Stable (STA) allografts were defined as allografts which had stable serum creatinine values, and absence of significant injury on pathology.

Sample collection and processing: Urine samples (50-100 mL) were collected, mid-stream, in sterile containers and then centrifuged at 2000×g for 20 min at room temperature within 1 h of collection. The supernatant was separated from the urine pellet containing cells and cell debris. The pH of the supernatant was adjusted to 7.0 using TrisHCl and stored at −80° C. until further analysis. Urine creatinine was measured using Quantichrom™ Creatinine Assay Kit (DICT-500) (BioAssay Systems, Hayward, Calif.). Total protein was measured for each urine sample using Coomassie Plus Beadford Assay Kit (Thermo Scientific, Rockford, Ill.).

Blood Samples: To avoid sequencing of donor and recipient DNA for evaluation of dd-cfDNA, a model of first evaluating 20 female recipients of male kidney donors was used which were then evaluated Chr Y cfDNA, which by extrapolation had to be donor derived. Blood samples were collected at the time of biopsy confirmed acute rejection (AR, n=4), and at the time of no AR (n=16); the no-AR samples were grouped into acute tubular necrosis (ATN, n=4), bacterial sepsis and pyelonephrotos (n=4), chronic allograft injury (CAI; n=4) and at stable graft function (n=4). Whole blood was collected into a sodium heparin tubes (BD Biosciences, San Jose, Calif.). Cells (lymphocytes) were removed using a Ficoll (Ficoll-Pague PLUS, GE Healthcare, Waukesha, Wis.) based density gradient centrifuge separation method, with the plasma fraction was pipetted off and stored frozen at −800 C until use.

Cell-free DNA extraction and quantification: cfDNA from urine and plasma samples was obtained using the QIAmp circulating nucleic acids kit (Qiagen, Valencia, Calif.), from 5 ml of urine and 3 ml of plasma, removed after total thawing at room temperature. As described in the manufacturer's protocol, these samples were first treated with proteinase K (supplied) to degrade cellular debris and remove DNases and RNases. Samples were then buffered and RNA carrier was added to assist in precipitation of cfDNA. This lysate was run through a DNA binding column, and then washed multiple times with buffers (supplied) and 100% ethanol, with the bound DNA eluted using 50 μl of buffer (supplied). The DNA containing eluent was used for DNA quantification and digital PCR (dPCR). 1 μl of eluent was used to quantify the double stranded cfDNA using the Quant-iT Pico Green kit (Invitrogen, Carlsbad, Calif.) in a 1/100 dilution with 1×TE buffer (supplied), as described in the manufacturers protocol. This was combined with an equal volume of a 1/200 dilution of Quant-iT reagent in each well (black microtiter plate) with the resulting fluorescence read with a spectrofluorometer (Gemini EM, Molecular Devices, Sunnyvale, Calif.). The concentration (ng/ml) for each sample was calculated by comparison to a 10-fold standard curve (lambda DNA, supplied).

Cell-free DNA measurement: Digital PCR (dPCR) was performed using 5 ng of extracted dd-cfDNA from urine on 12.765 digital array chips with the Biomark real-time PCR system (Fluidigm, South San Francisco, Calif.). Primers and labeled probes (IDT, Coralville, Iowa) were derived for the following: 1) for each locus of single copy Chromosome (Chr) Y for SRY (Forward primer: 5′CGCTTAACATAGCAGAAGCA; Reverse primer: 5′-AGTTTCGAACTCTCTGGCACCT; Probe: 5′TGTCGCACTCTCCTTGTTTTTGACA), 2) for each locus of multi-copy Chr Y DYS14 (Forward primer: 5′-ATCGTCCATTTCCAGAATCA; Reverse primer: 5′-GTTGACAGCCGTGGAATC; Probe: 5′-FAM-TGCCACAGACTGAACTGAATGATTTTC-BHQ1); 3) for Chr 1 Locus EIF2C1 (Forward Primer: 5′-GTTCGGCTTTCACCAGTCT; Reverse Primer: 5′-CTCCATAGCTCTCCCACTC; probe: 5′-HEX-CGCCCTGCCATGTGGAAGAT-BHQ1). The number of copies of each locus was calculated by the dPCR Analysis software v3.0 for copies per ml of sample extracted, and normalized against measured urine creatinine. ALU repeats were calculated by quantitative PCR (qPCR) using primers and labeled probes (IDT, Coralville, Iowa) (Forward primer: 5′-GCC TGT AAT CCC AGC TAC TC-3′; Reverse primer: 5′-ATC TCG GCT CAC TGC AAC-3′; Probe: 5′-5HEXTTCA AGC GAT TCT CCT GCC TCA GC 3BHQ1-3′). Standard protocols were used for qPCR reactions on the ABI ViiA7 (Applied Biosystems, Foster City, Calif.) under standard conditions (10 min at 95° C., 40 cycles of 15 s 95° C., 30 s at 60° C.). Human genomic DNA (Promega, WI) was used as a calibration standard for ALU repeats.

Statistical Analyses: Nominal clinical variables were analyzed using either an unpaired two tailed Students T-Test (parametric) or Mann-Whitney (non-parametric). Categorical clinical and histopathology data (Banff score ≧1) were analyzed using a Fisher exact test with two tailed p-value. Correlations were performed using a Spearman's Rank correlation coefficient, with correlations ≧0.3 and p-value <0.05 considered significant. A probability of less than 0.05 was considered significant for all statistical analyses, which were calculated using GraphPad Prism (Graphpad Software Inc., La Jolla, Calif.). All values are expressed as mean±standard deviation unless specified.

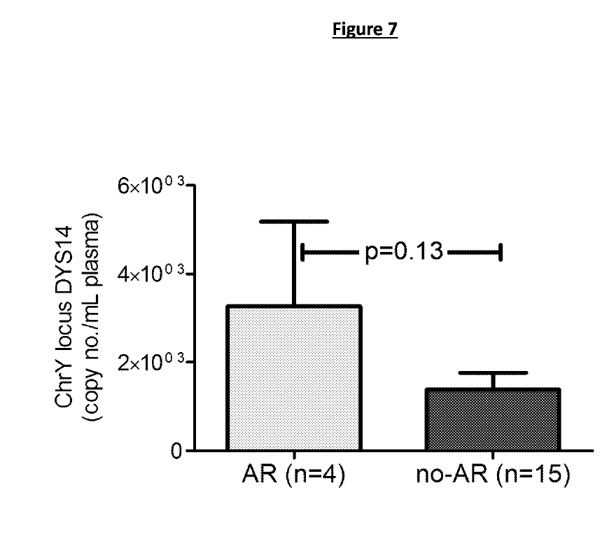

Plasma donor derived cell-free DNA (dd-cfDNA) is increased in acute kidney transplant injury: In the selected cohort of 19 female recipients of male grafts, there were greater quantities of plasma dd-cfDNA at the time of AR when compared to plasma dd-cfDNA values measured during stable (STA) graft function (mean copy number of Chr Y multi-copy locus DYS14/mL plasma was 3268±1917 for AR vs. 1385±378 for STA) (p=0.13) (FIG. 7). There was almost no signal for dd-cfDNA in plasma when the single locus Chr1 SRY was evaluated. When repeated for 3 separate plasma samples copy number detection of single locus SRY in the plasma we observed signal only from one of the three and average copy number calculated was 0.65±1.12 per μL loaded DNA sample whereas for the multi-locus DYS14 the copy number on the same samples was 82.82±73.52.

Analysis of dd-cfDNA in urine: As described earlier, the detection of single locus SRY was not possible in the plasma. Whereas, the signal for dd-cfDNA from the analysis of copy number of locus SRY of ChrY showed highest urinary load for biopsy confirmed AR, significant increase in urine dd-cfDNA load was also seen in other conditions with acute renal transplant injury such as ATN and BKVN.

Native renal tissue in a recipient with end stage renal disease is non-functional. With this in mind, single-locus EIF2C1 Chr 1 cfDNA (cfDNA load from an autosomal chromosome) was measured in the same 20 female recipients or male donor kidneys and the results correlated/sample to the results obtained from the urine Chr Ydd-cfDNA loads in the same samples as measured from the copy number of locus SRY.

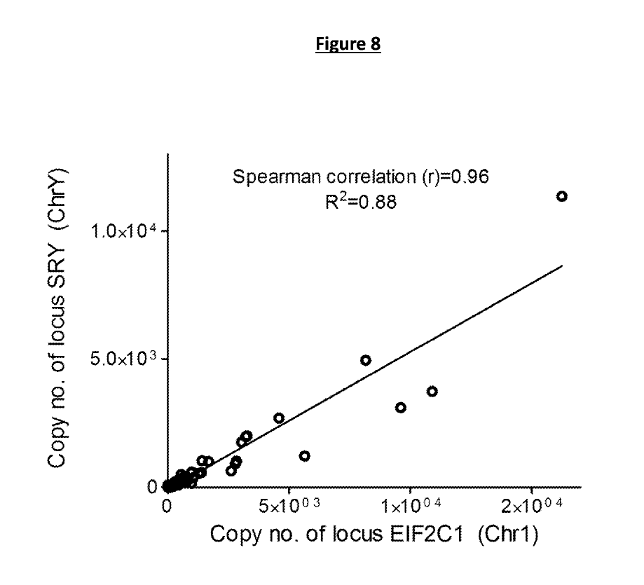

The urine cfDNA load as determined by the copy number of Chr 1 locus EIF2C1 analysis was almost entirely donor derived; as the urine cfDNA load for Chr Y locus SRY and Chr 1 locus EIF2C1 were highly correlated (r=0.96, R2 0.88, p value <0.0001; FIG. 8).

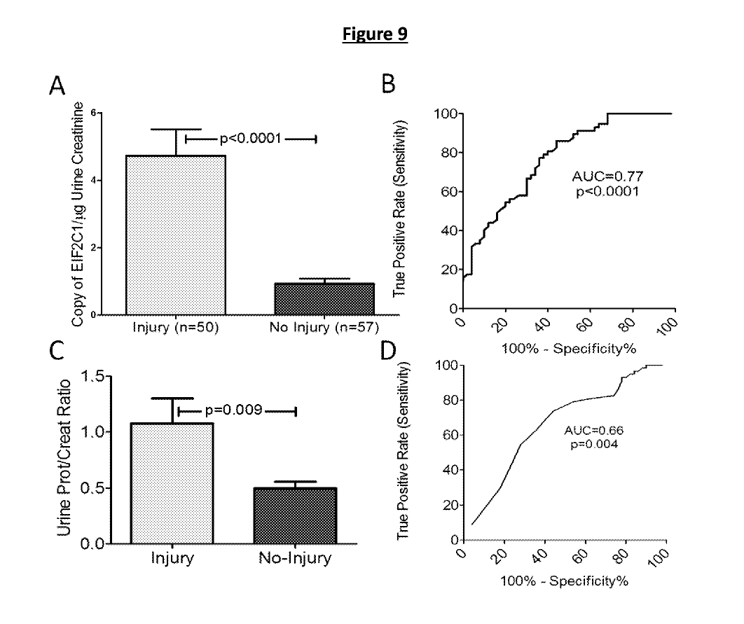

Urine Chr1 cfDNA copy number correlates with non-specific acute renal transplant injury: The urine Chr 1 cfDNA load was next measured in an independent cohort of 37 donor/recipient pairs of different gender combinations. There was a wider variation of Chr1 locus EIF2C1 with 0-21.57 copies/μg urine creatinine with a median of 0.97 copies across all samples irrespective of phenotype. The copy number of Chr1 locus EIF2C1 in the urine from AR patients was 4.87±1.22 copies/μg urine creatinine was significantly higher compared to the copy number of Chr1 locus EIF2C1 in the urine from no-injury phenotypes (STA) 0.93±0.15 (p<0.0001). However when the copy number of Chr1 locus EIF2C1 in the urine from AR patients (4.87±1.22 copies/μg urine creatinine) was compared with the copy number of Chr1 locus EIF2C1 in the urine from other injury phenotypes that included CAN and BKVN (4.77±1.07 copies/μg urine creatinine) there was no significant difference in the 0.93±0.15 with a P value of 0.94. The copy number of Chr1 locus EIF2C1 in the urine from acute injury phenotypes (for example, AR and BKVN) 4.72±0.78 copies/μg urine creatinine was significantly higher compared to the copy number of Chr1 locus EIF2C1 in the urine from no-injury phenotypes (STA) 0.93±0.15 with a P value of <0.0001 (FIG. 9A). ROC analysis showed that AUC of ROC curve for injury and no-injury phenotype as 0.77 with a p-value of <0.0001. At a threshold of 1.15 copy/μg urine creatinine, the true positive rate (sensitivity) was 77% with 64% specificity.

When dPCR was carried out for Chr 1 in the plasma, the data showed that autosomal plasma cfDNA was an admixture of donor and recipient cfDNA. Value of copy number of autosomal chromosome in the plasma as a marker for transplant injury was assessed by measuring Chr1 locus EIF2C1 copy number in the plasma of patients with transplant injury and no-injury. The copy number Chr1 locus EIF2C1 in the plasma of AR patients 12.34±0.97 was not significantly different than copy number 10.25±0.71 of Chr1 locus EIF2C1 in the plasma of patients with no-AR (p=0.13). The copy number Chr1 locus EIF2C1 in the plasma of AR patients was not significantly different than copy number of Chr1 locus EIF2C1 in the plasma of patients with chronic injury. The same was the case for the copy number Chr1 in the plasma of AR patients with injury vs the copy number of Chr1 in the plasma of patients with no-injury.

Urine cfDNA performs better than urine protein to creatinine ratio as an acute renal transplant injury marker: Proteinuria is a good marker of graft dysfunction. The same set of urine samples were analyzed for total protein to creatinine ratio. Urine protein to creatinine ratio in the samples from transplant injury was higher in injury group (1.08±0.22) when compared to non-injury group (0.50±0.06) with p-value 0.009. A ROC analysis on urine protein to creatinine ratio resulted in a ROC with AUC of 0.66 and p-value 0.004. At a threshold of 0.45 μg/μg urine creatinine, the true positive rate (sensitivity) was only 63% with 64% specificity (FIG. 9). When protein to creatinine ration was combined with Chr1 locus EIF2C1 copy number data there was an improved AUC of 0.82 with p<0.0001 and 79% sensitivity and 68% specificity.

Urine cfDNA correlates with renal transplant histological injury and renal function: To evaluate if the load of urine cfDNA is functionally correlated with intragraft histological injury and functional perturbation, irrespective of the injury Banff classification, urine dd-cfDNA (measured by dPCR of Chr Y and Chr 1) was correlated from AR with different Banff graded semi-quantitative histological parameters of glomerular, tubular and interstitial injury of the time matched blinded biopsy histology and estimated glomerular filtration rate (eGFR). Urine dd-cfDNA copy numbers/mcg of creatinine significantly correlated with eGFR (p=7.70E-03, r=−0.28) and on the matched biopsy histology markers of inflammation (i-score; p=1.2E-03, r=0.32), the tubulitis score (t-score; p=1.3E-03, r=0.32), and the glomerular inflammation score (g-score; 2.30E-02, r=0.23).

The burden of dPCR for urine dd-cfDNA correlates with qPCR measured urine ALU repeats: Total chromosomal DNA was measured in urine by measuring the ALU repeats from cfDNA. Dd-cfDNA measurement by Chr Y and Chr 1 was correlated with total urine cfDNA as estimated by the ALU repeat QPCR assay (r=0.87; p<0.0001). Due to this correlation in between dd-cfDNA as estimated by ChrY and total cfDNA by ALU repeat, ALU repeat QPCR was conducted on an independent set of urine samples and the ALU repeat load was correlated with the sample phenotype as assessed by the matched allograft biopsy Banff score.

Urine total cfDNA as estimated by ALU repeat QPCR is an indicator of acute kidney transplant injury: Genome DNA copy number in the urine based on ALU elements was calculated for all sample phenotypes. Based on the genome equivalents (GE) calculated from ALU assay data, there was an increased load of cfDNA in the urine of stable transplant patients (2.06±5.75 GE/mg urine creatine) when compared to healthy normal controls with normal renal function (0.11±0.18 GE/mg urine creatine) (p=4.25×10-07). The increase in ALU repeats was further significantly higher in urine samples collected at the time of biopsy confirmed AR (10.55±25.43 GE/mg urine creatine) when compared to patients with stable graft function and normal graft histology on protocol biopsy (STA; (2.06±5.75 GE/mg urine creatine)) (p=0.002). The increase in urine cfDNA for other acute injury phenotypes such as BKVN (6.55±7.02 GE/mg urine creatine) was also significantly higher than the values in STA patients (p=0.04). However the increase of cfDNA load in patients with chronic and other graft injury phenotypes (5.85±23.24) was higher than patients with stable graft function and normal graft histology on protocol biopsy (STA; (2.06±5.75 GE/mg urine creatine) but not statistically different (p=0.14).

The ROC analysis of the cfDNA data based on ALU assay of transplant injury vs no transplant injury showed an AUC of 0.73 with a p value 0.001.

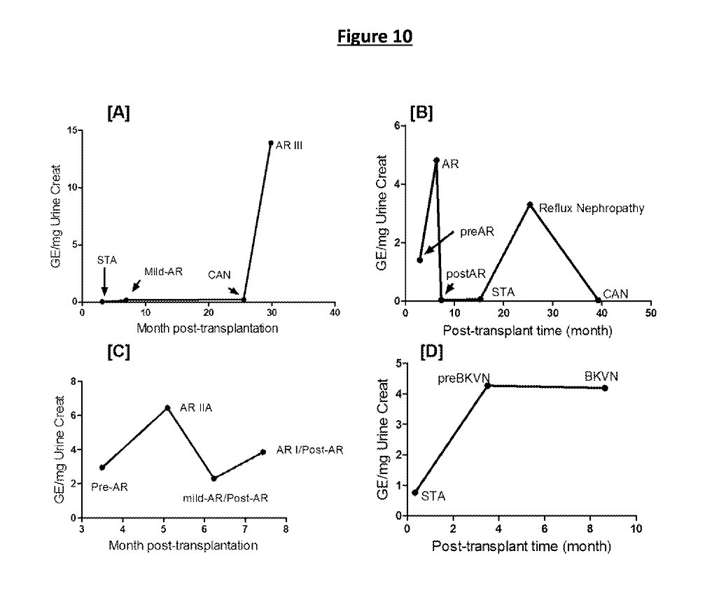

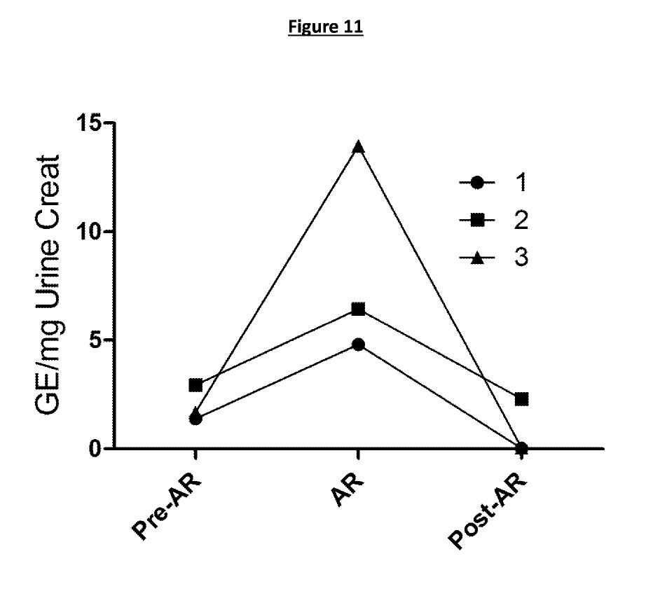

Serial analysis of urine cfDNA is a sensitive monitoring tool for prediction of transplant injury: The potential use of cfDNA load in the urine was evaluated by analyzing longitudinal urine samples collected from different patients. In all cases a spike was observed in the load of cfDNA at the time of acute injury such as AR and BKVN (FIG. 10). There was an increased load of cfDNA in the urine of severe AR when compared to low grade AR or other chronic injuries (FIG. 10A). The increase was observed in every AR event and other cases of acute injuries such as reflux nephropathy (FIG. 10B) and compared to stable graft function and other chronic injuries. There was a significant rise in cfDNA load in higher grade of AR (II and III) compared to border line and lower grade AR such as AR I (FIG. 10C). The increase in cfDNA load was higher in BKVN and the rise in cfDNA load could be observed in pre-BKVN urine (FIG. 10D). The rise in cfDNA load in the urine was observed as increased in AR episode when compared to pre- and post-AR following the same patients. Three patients (patient #1, #2, and #3) were evaluated for total cfDNA in the urine pre and post-AR which demonstrated an elevation of cfDNA load in the urine during AR episode (FIG. 11).

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A method for assessing renal status in an individual comprising:
a. determining the copy number of an autosomal chromosome in a urine sample, and
b. comparing the copy number of said chromosome to a standard copy number of said chromosome in a urine sample from a normal population,
wherein a change in the copy number is indicative of an altered renal status.

2. The method of claim 1 wherein a copy number determined to be higher than the standard copy number is indicative of compromised renal status.

3. The method of claim 2 wherein a compromised renal status comprises renal damage, renal injury, a renal disease, a renal disorder, renal graft rejection, or being non-responsive to a treatment for renal damage, renal injury, renal disease, renal disorder, or renal graft rejection.

4. The method of claim 1 wherein the assessment of renal status comprises measuring the progression of a renal disease, a renal injury, a renal graft injury, or a renal graft rejection.

5. The method of claim 1 wherein the assessment of renal status comprises measuring treatment response in and individual who is suffering from a renal disease, a renal injury, a renal graft injury, or a renal graft rejection and is currently undergoing or has undergone treatment.

6. The method of claim 1 wherein a copy number determined to be equal or lower than the standard copy number is indicative good renal health.

7. The method of claim 1, wherein cell free DNA is extracted from the urine.

8. The method of claim 1, wherein the copy number is determined in cell free DNA extracted from the urine.

9. The method of claim 1, wherein the copy number is determined using real-time PCR, quantitative PCR, or digital PCR.

10. The method of claim 9, wherein the copy number is determined using the BioMark real-time PCR system.

11. The method of claim 1, wherein the autosomal chromosome is chromosome 1.

12. The method of claim 11 wherein the copy number of chromosome 1 is measured using the EIF2C1 locus.

13. The method of claim 8 wherein the EIF2C1 locus is measured using a primer set comprising a forward primer 5′-GTTCGGCTTTCACCAGTCT and a reverse primer 5′-CTCCATAGCTCTCCCACTC.

14. The method of claim 13 wherein the primer set is utilized for real-time PCR and further includes a probe.

15. The method of claim 14 wherein the probe comprises the sequence and reporters as follows: 5′-HEX-CGCCCTGCCATGTGGAAGAT-BHQ1.

16. The method of claim 1, further comprising measurement of other pathological and clinical data.

17. The method of claim 16 wherein the method comprises measuring the amount of creatinine.

18. The method of claim 16 wherein the method comprises measuring proteinuria or cGFR.

19. The method of claim 16 wherein the method comprises performing a renal biopsy.

20. The method of claim 1 wherein the individual is an individual at risk for renal damage, renal injury, a renal disease, a renal disorder, renal graft injury, or renal graft rejection.

21. The method of claim 1 wherein the individual is diabetic.

22. The method of claim 1 wherein the individual suffers from hypertension.

23. The method of claim 1 wherein the individual is a recipient of an allograft renal transplant.

24. The method of claim 1 wherein the individual is under treatment for renal graft injury or renal graft rejection.

25. The method of claim 1 wherein the individual has suffered at least one acute rejection episode.

26. A method for assessing renal status in an individual comprising:
a. determining the number of ALU repeats in a urine sample, and
b. comparing the number of ALU repeats to a standard number of ALU repeats in a urine sample from a normal population,
wherein a change in the number of ALU repeats is indicative of an altered renal status.

27. The method of claim 26 wherein a number of ALU repeats determined to be higher than the standard number of repeats is indicative of compromised renal status.

28. The method of claim 27 wherein a compromised renal status comprises renal damage, renal injury, a renal disease, a renal disorder, renal graft rejection, or being non-responsive to a treatment for renal damage, renal injury, renal disease, renal disorder, or renal graft rejection.

29. The method of claim 26 wherein the assessment of renal status comprises measuring the progression of a renal disease, a renal injury, a renal graft injury, or a renal graft rejection.

30. The method of claim 26 wherein the assessment of renal status comprises measuring treatment response in and individual who is suffering from a renal disease, a renal injury, a renal graft injury, or a renal graft rejection and is currently undergoing or has undergone treatment.

31. The method of claim 26 wherein the number of ALU repeats determined to be equal or lower than the standard number of ALU repeats is indicative good renal health.

32. The method of claim 26, wherein cell free DNA is extracted from the urine.

33. The method of claim 26, wherein the number of ALU repeats is determined in cell free DNA extracted from the urine.

34. The method of claim 26, wherein the number of ALU repeats is determined using real-time PCR, quantitative PCR, or digital PCR.

35. The method of claim 9, wherein the number of ALU repeats is determined using the BioMark real-time PCR system.

36. The method of claim 26, wherein the number of ALU repeats is determined using real-time PCR.

37. The method of claim 36 wherein the number of ALU repeats is measured using a 115 base pair amplicon of the ALU locus.

38. The method of claim 26, wherein the method further comprises measuring the number of copies of an autosomal chromosome in the same sample.

39. The method of claim 38 wherein the copy number of chromosome 1 is measured.

40. The method of claim 39 wherein the copy number of Chromosome 1 is measured using the EIF2C1 locus.

41. The method of claim 40 wherein the EIF2C1 locus is measured using a primer set comprising a forward primer 5′-GTTCGGCTTTCACCAGTCT and a reverse primer 5′-CTCCATAGCTCTCCCACTC.

42. The method of claim 41 wherein the primer set is utilized for real-time PCR and further includes a probe.

43. The method of claim 42 wherein the probe comprises the sequence and reporters as follows: 5′-HEX-CGCCCTGCCATGTGGAAGAT-BHQ1.

44. The method of claim 26, further comprising measurement of other pathological and clinical data.

45. The method of claim 44 wherein the method comprises measuring the amount of creatinine.

46. The method of claim 44 wherein the method comprises measuring proteinuria or cGFR.

47. The method of claim 44 wherein the method comprises performing a renal biopsy.

48. The method of claim 26 wherein the individual is an individual at risk for renal damage, renal injury, a renal disease, a renal disorder, renal graft injury, or renal graft rejection.

49. The method of claim 26 wherein the individual is diabetic.

50. The method of claim 26 wherein the individual suffers from hypertension.

51. The method of claim 26 wherein the individual is a recipient of an allograft renal transplant.

52. The method of claim 26 wherein the individual is under treatment for renal graft injury or renal graft rejection.

53. The method of claim 26 wherein the individual has suffered at least one acute rejection episode.

54. A diagnostic assay kit, the kit comprising:
a. reagents for determining the copy number of at least one autosomal chromosome from a sample;
b. a primer set used for determining said copy number; and
c. instructions for use of the assay.

55. The kit of claim 54 further comprising reagents for extracting cell free DNA from a sample.

56. The kit of claim 54 wherein the sample comprises urine.

57. The kit of claim 54 wherein autosomal chromosome is Chromosome 1.

58. The kit of claim 57 wherein the primer set is a set capable of amplifying an amplicon of locus EIF2C1.

59. The kit of claim 58 wherein the primer set comprises the forward primer 5′-GTTCGGCTTTCACCAGTCT and the reverse primer 5′-CTCCATAGCTCTCCCACTC.

60. The kit of claim 54 wherein the primer set is capable of being used for real-time PCR, quantitative PCR, or digital PCR.

61. The kit of claim 59 wherein the primer set additionally comprises a probe useful for digital PCR.

62. The kit of claim 61 wherein the sequence of the probe comprises the sequence and reporters as follows: 5′-HEX-CGCCCTGCCATGTGGAAGAT-BHQ1.

63. A diagnostic assay kit, the kit comprising:
a. reagents for determining the number of ALU repeats in a sample;
b. a primer set used for determining said number of ALU repeats; and
c. instructions for use of the assay.

64. The kit of claim 63 further comprising reagents for extracting cell free DNA from a sample.

65. The kit of claim 64 wherein the sample comprises urine.

66. The kit of claim 63 wherein the primer set is a set capable of amplifying a 115 base pair amplicon of the ALU locus.

67. The kit of claim 66 wherein the primer set comprises the forward primer 5′-GCCTGTAATCCCAGCTACTC-3′ and the reverse primer 5′-ATCTCGGCTCACTGCAAC-3′.

68. The kit of claim 63 wherein the primer set is capable of being used for real-time PCR, quantitative PCR, or digital PCR.

69. The kit of claim 67 wherein the primer set additionally comprises a probe useful for digital PCR.

70. The kit of claim 69 wherein the sequence of the probe comprises the sequence and reporters as follows: 5′-HEXTCAAGCGATTCTCCTGCCTCAGC-BHQ-3′.

71. The kit of claim 63 wherein the kit additional comprises a primer set capable of measuring the copy number of an autosomal chromosome.

72. The kit of claim 71 wherein the autosomal chromosome is Chromosome 1.

73. The kit of claim 72 wherein the primer set is a set capable of amplifying an amplicon of locus EIF2C1.

74. The kit of claim 73 wherein the primer set comprises the forward primer 5′-GTTCGGCTTTCACCAGTCT and the reverse primer 5′-CTCCATAGCTCTCCCACTC.

75. The kit of claim 74 wherein the primer set is capable of being used for real-time PCR, quantitative PCR, or digital PCR.

76. The kit of claim 74 wherein the primer set additionally comprises a probe useful for digital PCR.

77. The kit of claim 76 wherein the sequence of the probe comprises the sequence and reporters as follows: 5′-HEX-CGCCCTGCCATGTGGAAGAT-BHQ1.
